(19) United States
(12) Patent Application Publication
(10) Pub. No.: US 2004/0048250 A1
(43) Pub. Date: Mar. 11, 2004

(54) Gene encoding abc-1 paralogs and the polypeptides derived therefrom

(76) Inventors: Margaret Ann Johns, Ann Arbor, MI (US); Sherrie Rae Tafuri, Ann Arbor, MI (US); Minghan Wang, Ballwin, MO (US)

Correspondence Address: Cynthia M Bott, Warner Lambert Company, 2800 Plymouth Road, Ann Arbor, MI, 48105, US

(21) Appl. No.: 10/182,006

(22) PCT Filed: Jan. 23, 2001
(86) PCT No.: PCT/US01/02191

(51) Int. Cl. C12Q 1/68; C07H 21/04; C12P 21/02; C12N 5/06; C07K 14/47

(52) U.S. Cl. 435/6; 435/69.1; 435/320.1; 435/325; 530/350; 536/23.5; 514/44

(57) ABSTRACT

The present invention relates to three novel genes and polypeptides derived therefrom encoding “PD-ABC” proteins. The invention also describes methods for using the novel gene and polypeptides in the detection of genetic deletions of the gene, subcellular localization of the polypeptide, binding assays in connection with the chemical databases, gene therapy, and identification of chemicals which may be used in the therapeutic treatment of PD-ABC-mediated diseases.

### FIELD OF THE INVENTION

The present invention relates to the identification of the PD-ATP-binding cassette gene (hereinafter “PD-ABC gene”) and polypeptides derived and identified therefrom, and use of the PD-ABC genes for drug screening assays, and diagnostic and therapeutic methods for the treatment of cardiovascular and inflammatory disorders, mediated by the expression of a mutant form or by aberrant levels or activity of the PD-ABC genes. The invention is based on the discovery that the PD-ABC gene sequences encode polypeptides that are paralogous to the ATP-binding cassette transporter gene family, the human gene localizes to a chromosomal region implicated in cardiovascular disease and abnormal HDL metabolism, and the human gene is expressed in cells implicated in cardiovascular and inflammatory diseases. In particular, human novel PD-ABC gene sequences and polypeptides derived and identified therefrom encoding human PD-ABC polypeptides are disclosed. In addition, the chromosomal localization of PD-ABC to human chromosome 19p13.3 and the expression of PD-ABC in spleen, thymus, peripheral blood leukocytes, bone marrow, lymph nodes, and additional tissues is disclosed. The invention also describes vectors and host cells comprising the PD-ABC genes and methods for using the PD-ABC genes, polypeptides, and antibodies specifically targeting the polypeptides in the detection of genetic alterations of the PD-ABC genes, subcellular localization of the polypeptides, gene therapy applications, or binding assays in connection with chemical databases. The invention also relates to the development of proprietary screening strategies for molecules which modify PD-ABC protein activities, diagnostics for syndromes associated with altered PD-ABC protein expression, and methods for the identification of compounds that modulate the expression, synthesis, or activity of the PD-ABC genes/proteins and to using those compounds such as those identified as therapeutic agents in the treatment of PD-ABC mediated disorders; including by way of example and not of limitation, coronary artery disease (CAD).

### BACKGROUND OF THE INVENTION

ATP-binding cassette (ABC) transporters constitute a large family of transmembrane proteins which transport a wide variety of substrates across cell membranes (Higgins, C. F., Annu. Rev. Cell Biol., 1992;8:67-113). Members of this transporter family have two hydrophobic domains, each containing six transmembrane segments. In addition, they have two cytoplasmic ATP-binding cassettes or nuclear binding folds (NBF) at the carboxyl terminus of each hydrophobic domain. ATP binding and hydrolysis at the NBF provides energy for transport activity (Higgins, C. F., Annu. Rev. Cell Biol., 1992;8:67-113). Although different members share significant homology, ATP transporters have diverse substrate specificities. The multidrug-resistant p-glycoprotein (MDR) transports organic chemicals with unrelated structures while the related transporter MRP is associated with membrane translocation of phospholipid (Gottesman, M. M., et al., Annu. Rev. Biochem., 1993;62:385-428; Smit, J. J., et al., Cell, 1993;75:451-462; Ruetz, S., et al., Cell, 1994;77:1071-1081). The physiological importance of the ABC transporters has been highlighted by findings that genetic defects in some ABC transporters are linked to human diseases. Mutations in the cystic fibrosis transmembrane conductance regulator gene are the cause of cystic fibrosis (Riordan, J R., et al., Science, 1989;245:1066-1073). Genetic mutations or truncations in ABCR, an ABC transporter of the ABCA (Broccardo, C., et al., Biochim. Biophys. Acta, 1999; 1461:395-404), result in Stargardt Disease, a degenerative retina illness (Allikmets, R., et al., Nat. Genet., 1997; 15:236-246). ABC1, also a member of the ABCA subfamily, and was isolated from mouse by PCR based on sequence homology (Luciani, M -F., et al., Genomics, 1994;21:150-159). ABC1 was initially found to be required for the engulfment of apoptotic cells and for anion transport across membranes (Luciani, M -F., et al., EMBO J., 1996;15:226-235; Becq, F., et al., J. Biol. Chem., 1997;272:2695-2699.). The expression of human ABC1 is regulated during macrophage differentiation and by cholesterol loading (Langmann, T., et al., Biochem. Biophys. Res. Comm., 1999;257:29-33). Recently ABC1 was identified as the defective gene in Tangier Disease (TD), a rare form of lipoprotein deficiency (Bodzioch, M., et al., Nature Genetics, 1999;22:347-351; Broccardo, C., et al., Biochim. Biophys. Acta, 1999;1461:395-404; Brooks-Wilson, A., et al., Nature Genetics, 1999;22:336-345; Rust, S., et al., Nature Genetics, 1999;22:352-355). Both genetic and pharmacological evidence suggest that ABC1 transports cholesterol and phospholipid across the cell membrane in peripheral tissues (Remaley, A. T., et al., Proc. Natl. Acad. Sci. USA, 1999;96:12685-12690; Young, S. G., et al., Nature Genetics, 1999;22:316-318). A phenotype similar to that in TD patients was observed in ABC1 knockout mice (McNeish, J., et al., Proc. Natl. Acad. Sci. USA, 2000;97:4245-4250; Orso, E., et al., Nature Genetics, 2000;24:192-196).

The implication of ABC1 in lipoprotein metabolism led us to search for close homologues of ABC1 that might be involved in cholesterol efflux and reverse cholesterol transport. Here we describe the isolation and tissue-specific expression of a novel ABC transporter that is the closest ABC1 homologue to date. Further, we also identified an alternatively spliced variant that has a different tissue-specific expression pattern. These findings suggest that PD-ABC might have an important physiological role.

An increased risk for CAD has been associated with a low level of high-density lipoprotein (HDL) particles. In fact, nearly one-half of all patients with CAD have low levels of HDL cholesterol. As a result, recent efforts for prevention of CAD have focused on methods of increasing the levels of HDL particles. HDL particles are important for ridding the body of excess cholesterol by transporting cholesterol from cells to the liver (reverse cholesterol transport), where cholesterol metabolism and eventual excretion take place. Tangier's disease and familial high-density lipoprotein deficiency (FHA) are characterized by extremely low plasma levels of HDL and increased levels of cellular cholesterol, with resulting premature atherosclerosis (Rogler, et al., Arterioscler Thromb Vasc Biol, 1995;15(5):683-90; Marcil, et al., Arterioscler Thromb Vasc Biol., 1999;19(1):159-69). Mutations in the to ATP-binding cassette transporter-1 (ABC1) gene were recently identified in TD and FHA patients (Brooks-Wilson, A., et al., Nature Genetics, 1999;22:336-345; Bodzioch, M., et al., Nature Genetics, 1999;22:347-351). These studies show that the ABC1 protein plays a critical role in the efflux of cholesterol from cells into HDL particles (Marcil, et al., Lancet, 1999;354:1340-1346). It has become clear that increasing the activity of the ABC1 protein, through the use of small molecule compounds, may be one way to raise HDL levels and to prevent CAD.

Although the ABC1 gene/protein was recently identified as playing a role in HDL metabolism, it is clear that other genes will also be involved. These other genes have not yet been identified. It would be beneficial if such genes were to be identified. By gaining an understanding of the biochemical mechanisms behind this pathway, new opportunities for treating and diagnosing diseases related to abnormal (high or low) production of HDL particles, may be achieved. Stated another way, a better understanding of the molecular mechanisms of HDL-mediated efflux of intracellular cholesterol will allow improved design of therapeutic drugs that treat diseases related to abnormal levels of cholesterol and production of HDL.

Dyslipidemia, such as alterations in HDL metabolism, or CAD resulting from dyslipidemia has been associated with a number of diseases. Such diseases includes diabetes (for review, Evans, et al., Curr Opin Lipidol, 1999;10(5):387-391), fatty liver disease (Marchesini, et al., Am J Med, 1999;107(5):450-455), obesity (for review, Indulski, et al., Cent Eur J Public Health, 1999;7(3):122-129), insulin resistance (for review, Bailey, et al., Biochem Pharmacol, 1999;58(10):1511-1520), alcoholism (for review, Baraona, et al., Recent Dev Alcohol, 1998;14:97-134), retinal degeneration (Gordon, et al., Am J Ophthalmol, 1991;112(4):385-391), hypertension (for review, Giannattasio, et. al., Pathol Biol (Paris), 1999;47(7):744-751), and vascular diseases in general.

### SUMMARY OF THE INVENTION

In order to determine if other genes exist which may also be important in the regulation of cholesterol levels, commercially available sequence databases were searched for genes related to 1 (ATP-binding cassette transporter-1 hereinafter “ABC1”). The ABC transporter gene family is the largest known gene family, and the ABC transporter genes have diverse substrates including sugars, amino acids, peptides, and antibiotics (for review, Croop J M, Methods Enzymol, 1998;292:101-116). By performing homology searches of a proprietary database of clustered EST sequences, generated using algorithms and tools from Compugen Systems, Ltd., novel human gene sequences were identified which are 48% identical and 64% similar at the amino acid level to ABC1. These genes (hereinafter referred to as PD-ABC Form 1 and Form 2) represent the closest human paralogs of ABC1 gene so far identified. The PD-ABC genes, and the polypeptides they encode are expressed in various cells and tissues, and are identified herein, both the polynucleotide sequences for the full length genes and any splice variants and their encoded proteins. The polynucleotide sequence of PD-ABC Form 1 is identified in SEQ ID NO 1 and the amino acid sequences of the PD-ABC Form 1 protein encoded by the novel gene is set forth in SEQ ID NO 2. The polynucleotide sequence of PD-ABC Form 2 is identified in SEQ ID NO 3 and the amino acid sequences of the PD-ABC protein encoded by the novel gene is set forth in SEQ ID NO 4.

Additionally, alignment of these genes to high-throughput genomic sequences in the Genbank database led to the “in silico” localization of these genes to human chromosome 19p 13.3. Searches of the Online Mendelian Inheritance in Man (OMIM) database led to the discovery that this same region of human chromosome 19 has been genetically linked to atherosclerosis susceptibility (Nishina, et al., PNAS, 1992;89:708-712; Naggert, et al., Clin Genet, 1997:236-240). Affected individuals from families which show linkage to 19p13.3 have low levels of HDL particles, characteristic of mutations in ABC1 gene. The level of identity of these novel genes to ABC1 gene and genetic linkage to a locus implicated in atherosclerosis susceptibility, identify these genes as a target for drugs to prevent CAD. In other words, because PD-ABC Form land 2 proteins shares amino acid homology to ABC1 protein, it is very likely that they share some structural and functional characteristics with ABC1.

One aspect of the invention is to provide purified PD-ABC Form 1 and 2 proteins. The purified proteins may be obtained from either recombinant cells or naturally occurring cells. The purified PD-ABC proteins of the invention may be mammalian in origin. Primate, including human-derived PD-ABC proteins, are examples of the various proteins specifically provided for. The invention also provides allelic variants and biologically active derivatives of naturally occurring PD-ABC proteins.

Another aspect of the invention is to provide polynucleotides encoding the PD-ABC Form 1 and 2 proteins of the invention and to provide polynucleotides complementary to polynucleotide coding strand. The polynucleotides of the invention may be used to provide for the recombinant expression of PD-ABC proteins. The polynucleotides of the invention may also be used for genetic therapy purposes so as to 1) treat diseases which may result from alterations of PD-ABC genes or from alterations of cellular pathways involving PD-ABC genes/proteins, 2) test for presence of a disease, or susceptibility to a disease, due to alterations or deletions PD-ABC genes/proteins, 3) analyze or alter the subcellular localization of the PD-ABC polypeptides, 4) clone or isolate discrete classes of RNA similar to PD-ABC genes, 5) express discrete classes of RNA in order to alter the levels of PD-ABC genes.

The invention also relates to oligonucleotide molecules useful as probes or primers, wherein said oligonucleotide molecules hybridize specifically with any nucleotide sequence comprising or related to the PD-ABC genes, particularly the sequences of SEQ ID NOS 1 and 3. These oligonucleotides are useful either as primers for use in various processes such as DNA amplification and microsequencing or as probes for DNA recognition in hybridization analyses.

A nucleic acid probe or primer according to the invention comprises at least 8 consecutive nucleotides of a polynucleotide of SEQ ID NOS 1 or 3, preferably from 8 to 200 consecutive nucleotides, more particularly from 10, 15, 20, or 30 to 100 consecutive nucleotides, more preferably from 10 to 90 nucleotides, and most preferably from 20 to 80 consecutive nucleotides of a polynucleotide of SEQ ID NOS 1 or 2. Preferred probes or primers of the invention comprise the oligonucleotides selected from the group consisting of the oligonucleotides set forth in the examples below.

The invention also concerns a method for the amplification of a region of the PD-ABC genes. The method comprises the step of: contacting a test sample suspected of containing the desired PD-ABC sequences or portions thereof with amplification reaction reagents, comprising a pair of amplification primers such as those described above, the primers being located on either side of the PD-ABC nucleotide regions to be amplified. The method may further comprise the step of detecting the amplification product. For example, the amplification product may be detected using a detection probe that can hybridize with an internal region of the amplified sequences. Alternatively, the amplification product may be detected with any of the primers used for the amplification reaction themselves, optionally in a labeled form.

The invention also concerns diagnostic kits for detecting the presence of at least one copy of a PD-ABC Form 1 or Form 2 DNA in a test sample, said kits containing a primer, a pair of primers or a probe according to the invention.

In a first embodiment, the kit comprises primers such as those described above, preferably forward and reverse primers which are used to amplify PD-ABC genes or fragments thereof.

In a second embodiment, the kit comprises a hybridization DNA probe, that is or eventually becomes immobilized on a solid support, which is capable of hybridizing with a PD-ABC gene or a fragment thereof. The techniques for immobilizing a nucleotide primer or probe on a solid support are well-known to the skilled person.

The kits of the present invention can also comprise optional elements including appropriate amplification reagents such as DNA polymerases when the kit comprises primers, reagents useful in hybridization reactions and reagents useful to reveal the presence of a hybridization reaction between a labeled hybridization probe and a PD-ABC gene.

Another aspect of the invention is to provide antibodies capable of binding to PD-ABC proteins of the invention. The antibodies may be polyclonal or monoclonal. The invention also provides methods of using the subject antibodies to detect and measure expression of PD-ABC proteins either in vitro or in vivo, or for detecting proteins that interact with PD-ABC proteins, or molecules that regulate any of the activities of PD-ABC proteins.

Another aspect of the invention is to provide assays for the detection of proteins that interact with PD-ABC genes/proteins using genetic approaches. A preferred embodiment involves the use of yeast two-hybrid approaches for this screening. (Bartel and Fields, The Yeast Two-Hybrid System, Oxford University Press, 1997)

Another aspect of the invention is to provide assays for the detection or screening of therapeutic compounds that interfere with, or mimic in any way, the interaction between PD-ABC proteins and ligands that bind PD-ABC proteins.

In a first embodiment, such a method for the screening of a candidate substance comprises the following steps:

a) providing a polypeptide comprising the amino acid sequence of SEQ ID NO 2 or 4, or a peptide fragment or a variant thereof;

b) obtaining a candidate substance;

c) bringing into contact said polypeptide with said candidate substance; and

d) detecting the complexes formed between said polypeptide and said candidate substance.

In one embodiment of the screening method defined above, the complexes formed between the polypeptide and the candidate substance are further incubated in the presence of a polyclonal or a monoclonal antibody that specifically binds to a PD-ABC protein of the invention or to the peptide fragment or variant thereof.

The candidate substance or molecule to be assayed for interacting with the PD-ABC polypeptide may be of diverse nature, including, without being limited to, natural or synthetic organic compounds or molecules of biological origin such as polypeptides.

In another embodiment of the present screening method, increasing concentrations of a substance competing for binding to the PD-ABC protein with the considered candidate substance is added, simultaneously or prior to the addition of the candidate substance or molecule, when performing Step c) of said method. By this technique, the detection and optionally the quantification of the complexes formed between the PD-ABC protein or the peptide fragment or variant thereof and the candidate substance or molecule to be screened allows the one skilled in the art to determine the affinity value of said substance or molecule for said PD-ABC protein or the peptide fragment or variant thereof.

The invention also pertains to kits useful for performing the hereinbefore described screening method. Preferably, such kits comprise a PD-ABC protein having the amino acid sequence of SEQ ID NOS 2 or 4 or a peptide fragment or a variant thereof, and optionally means useful to detect the complex formed between the PD-ABC protein or its peptide fragment or variant and the candidate substance. In a preferred embodiment the detection means consist in monoclonal or polyclonal antibodies directed against the PD-ABC protein or a peptide fragment or a variant thereof.

The assays of the invention therefore comprise the step of measuring the effect of a compound of interest on binding between PD-ABC proteins and the ligands that bind to PD-ABC proteins. Binding may be measured in a variety of ways, including the use of labeled PD-ABC protein or labeled ligands

Another aspect of the invention is to provide assays for the discovery of proteins that interact directly or indirectly with PD-ABC proteins. The assays of the invention comprise a method for detecting such interactions in cells, or in biochemical assays. These interactions may be detected in a variety of ways, including the use of the cDNA encoding PD-ABC proteins, or PD-ABC proteins themselves, or fragments or modifications thereof.

In one preferred embodiment of the present invention, PD-ABC genes represent novel targets which can be used to develop high-throughput screens for identification of chemicals and interacting proteins which increase the activity of PD-ABCs. Ultimately, compounds which alter the activity of PD-ABC genes can be tested for efficacy in the prevention of CAD. The loci may also be of use for basic research and pharmacogenetic studies related to HDL metabolism.

In another preferred embodiment of the present invention, the protein product of PD-ABC genes may serve as novel therapeutic targets for treatment of CAD and dyslipidemia.

In another preferred embodiment of the present invention, a genetic model for studying CAD and dyslipidemia can be created by altering PD-ABC genes in animals such as mice.

In a further preferred embodiment of the present invention, polymorphisms in PD-ABC genes may identify members of the population at risk for CAD, and a genetic test could be created using the sequences of these genes to identify such people.

In a further preferred embodiment of the present invention, polymorphisms in the sequence of PD-ABC genes could be used to choose appropriate methods of therapy for CAD and dyslipidemia.

In a further preferred embodiment of the present invention, the sequence of PD-ABC genes could be used to create antisense RNA or antibody probes which could then be used for therapeutic treatment of CAD or dyslipidemia.

In a still further preferred embodiment of the present invention, the sequences of PD-ABC genes could be used to identify interacting genes, which themselves could serve as therapeutic targets.

In a still further preferred embodiment of the present invention, nucleotide sequences including and surrounding the PD-ABC genes could be used to identify factors which regulate the levels of these genes. These factors could become therapeutic targets for dyslipidemia and CAD.

In a still further preferred embodiment of the present invention, the protein product of the PD-ABC genes could be used to identify compounds which are selective for a particular member of the ABC transporter family.

Described herein are preferred sequences, polypeptides, and methods for making and using the invention. However, it is to be understood that the invention is not to be limited only to the particular sequences, polypeptides and methods described. The sequences, polypeptides and methodologies may vary, and the terminology used herein is for the purpose of describing particular embodiments. The foregoing is not intended and should not be construed as limiting the invention in any way since the scope of protection will ultimately depend upon the claims. Unless defined otherwise, all technical and scientific terms used herein have the same meaning as is commonly understood by one of skill in the art to which this invention belongs. All U.S. patents and all publications mentioned herein are incorporated in their entirety by reference thereto.

### BRIEF DESCRIPTION OF THE INVENTION

FIG. 1. Alignment of the predicted amino acid sequence of human PD-ABC to human ABC1 and ABCR. Amino acids are shown in single letter code. Identical residues across all sequences are highlighted in dark area and homologous residues are marked in shaded area. Dashes indicate gaps introduced into the sequences to maximize the alignment.

[image]

[image]

[image]

[image]

[image]

FIG. 2. Structures of the splice variants and the intron/exon organization. Diagram A depicts the intron and exon locations in each variants as well as alternative splice site. The transmembrane domains (TM1=N-terminal, TM2=C-terminal) and nucleotide binding fold (NBF) are indicated.

[image]

FIG. 3. Expression of PD-ABC in multiple tissues. A, Northern blot analysis of PD-ABC expression in various human tissues. A blot with mRNA from the indicated tissues was hybridized with a human PD-ABC probe and a GAPDH probe, respectively. The PD-ABC and GAPDH bands are indicated with arrows. B, Northern blot analysis of PD-ABC expression in tissues or cells of the immune system. Hybridization was carried out as mentioned above and the two forms of PD-ABC are indicated.

[image]

FIG. 4. Tissue distribution of PD-ABC splice variants. Rapid-Scan Gene Expression Panels were used as templates and reverse transcription-polymeradse chain reactions were run with primer pairs specific to the two variants, respectively. The PCR products were resolved on agarose gel. Lane1, fetal liver; lane 2, fetal brain; lane 3, bone marrow; lane 4, PBL (peripheral blood leukocytes); lane 5, skin; lane 6, prostate; lane 7, uterus; lane 8, ovary; lane 9, pancreas; lane 10, adrenal; lane 11, thyroid; lane 12, salivary; lane 13, placenta; lane 14, testis; lane 15, stomach; lane 16, muscle; lane 17, small intestine; lane 18, lung; lane 19, colon; lane 20, liver; lane 21, spleen; lane 22, kidney; lane 23, heart; lane 24, brain.

[image]

### DETAILED DESCRIPTION OF THE INVENTION

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), and Gene Transfer and Expression Protocols, pp. 109-128, ed. E. J. Murray, The Humana Press Inc., Clifton, N. J.) Sequence Analysis Primer (Gribskov, et al., 1994, Oxford University Press).

In one aspect, the present invention provides novel isolated and purified polynucleotides, hereinafter referred to as ATP-binding cassette transporter 1 paralog (PD-ABC) genes, encoding PD-ABC proteins, wherein the polynucleotide sequences are substantially similar to those shown in SEQ ID NOS 1 and 3 and the polypeptide sequences are substantially similar to those shown in SEQ ID NOS 2 and 4. The term “PD-ABC” is used broadly herein. Unless noted otherwise, the term “PD-ABC” include any natural mammalian-derived form of PD-ABC and the like. It is preferred that the terms PD-ABC include all mammals, including but not limited to primates and humans.

The polynucleotides provided for may encode PD-ABC proteins or portions thereof. The polynucleotides of the invention may be produced by a variety of methods including in vitro chemical synthesis using well-known solid phase synthesis technique, by cloning or combinations thereof. The polynucleotide of the invention may be derived from cDNA or genomic libraries. Persons of ordinary skill in the art are familiar with the degeneracy of the genetic code and may readily design polynucleotides that encode PD-ABC proteins that have either partial or polynucleotide sequence homology to naturally occurring polynucleotide sequences encoding PD-ABC proteins. The polynucleotides of the invention may be single stranded or double stranded. Polynucleotide complementary to polynucleotides encoding PD-ABC proteins are also provided.

Polynucleotides encoding an PD-ABC protein can be obtained from cDNA libraries prepared from tissue believed to possess PD-ABC protein or mRNA and to express it at a detectable level. For example, a cDNA library can be constructed by obtaining polyadenylated mRNA from a cell line known to express PD-ABC protein, and using the mRNA as a template to synthesize double stranded cDNA.

Libraries, either cDNA or genomic, are screened with probes designed to identify the gene of interest or the protein encoded by it. For cDNA expression libraries, suitable probes include monoclonal and polyclonal antibodies that recognize and specifically bind to an PD-ABC protein. For cDNA libraries, suitable probes include carefully selected oligonucleotide probes (usually of about 20-80 bases in length) that encode known or suspected portions of an PD-ABC protein from the same or different species, and/or complementary or homologous cDNAs or fragments thereof that encode the same or a similar gene, and/or homologous genomic DNAs or fragments thereof. Screening the cDNA or genomic library with the selected probe may be conducted using standard procedures as described in Chapters 10-12 of Sambrook, et al., Molecular Cloning: A Laboratory Manual, New York, Cold Spring Harbor Laboratory Press, 1989).

A preferred method of practicing this invention is to use carefully selected oligonucleotide sequences to screen cDNA libraries from various tissues. The oligonucleotide sequences selected as probes should be sufficient in length and sufficiently unambiguous that false positives are minimized. The actual nucleotide sequence(s) is/are usually designed based on regions of a PD-ABC gene that have the least codon redundance. The oligonucleotides may be degenerate at one or more positions. The use of degenerate oligonucleotides is of particular importance where a library is screened from a species in which preferential codon usage is not known.

The oligonucleotide must be labeled such that it can be detected upon hybridization to DNA in the library being screened. The preferred method of labeling is to use ATP (e.g., T32P) and polynucleotide kinase to radiolabel the 5′ end of the oligonucleotide. However, other methods may be used to label the oligonucleotide, including, but not limited to, biotinylation or enzyme labeling.

cDNAs encoding PD-ABC proteins can also be identified and isolated by other known techniques of recombinant DNA technology, such as by direct expression cloning or by using the polymerase chain reaction (PCR) as described in U.S. Pat. No. 4,683,195, in section 14 of Sambrook, et al., Molecular Cloning: A Laboratory Manual, second edition, Cold Spring Harbor Laboratory Press, New York, 1989, or in Chapter 15 of Current Protocols in Molecular Biology, Ausubel, et al. eds., Green Publishing Associates and Wiley-Interscience 1991. This method requires the use of oligonucleotide probes that will hybridize to DNA encoding an PD-ABC protein.

As defined herein, “substantially similar” includes identical sequences, as well as deletions, substitutions or additions to a DNA, RNA or protein sequence that maintain any biologically active portion thereof of the protein product and possess any of the conserved motifs. This includes, but is not limited to, any splice variants of PD-ABC which are found to exist. Preferably, the DNA sequences according to the invention consist essentially of the DNA sequence of SEQ ID NOS 1 or 3. These novel purified and isolated DNA sequences can be used to direct expression of the PD-ABC proteins and for mutational analysis of PD-ABC proteins' function.

Mutated sequences according to the invention can be identified in a routine manner by those skilled in the art using the teachings provided herein, and techniques well known in the art.

In a preferred embodiment, the present invention comprises a nucleotide sequence that hybridizes to the nucleotide sequence shown in SEQ ID NO 1 or 3 under high stringency hybridization conditions. As used herein, the term “high stringency hybridization conditions” refers to hybridization on a filter support at 65° C. in a low salt hybridization buffer to the probe of interest at 2×10⁸ cpm/μg for between about 8 to 24 hours, followed by washing in 1% SDS, 20 mM phosphate buffer and 1 mM EDTA at 65° C., for between about 30 minutes to 4 hours. In a preferred embodiment, the low salt hybridization buffer comprises between, 0.5% to 10% SDS, and 0.05M and 0.5 M sodium phosphate. In a most preferred embodiment, the low salt hybridization buffer comprises 7% SDS and 0.125M sodium phosphate.

As known in the art, numerous equivalent conditions may be employed to comprise either low or high stringency conditions. Factors such as the length and nature (DNA, RNA, base composition) of the sequence, nature of the target (DNA, RNA, base composition, presence in solution or immobilization, etc.), and the concentration of the salts and other components (e.g., the presence or absence of formamide, dextran sulfate and/or polyethylene glycol) are considered and the hybridization solution may be varied to generate conditions of either low or high stringency different from, but equivalent to, the above listed conditions.

The term “stringent conditions”, as used herein, is the “stringency” which occurs within a range from about Tm-5° C. (5° C. below the melting temperature (Tm) of the probe) to about 20° C. to 25° C. below Tm. As will be understood by those of skill in the art, the stringency of hybridization may be altered in order to identify or detect identical or related polynucleotide sequences.

The polynucleotides of the invention have a variety of uses, some of which have been indicated or will be addressed in greater detail, infra. The particular uses for a given polynucleotide depend, in part, on the specific polynucleotide embodiment of interest. The polynucleotides of the invention may be used as hybridization probes to recover PD-ABC nucleotide sequences from genetic libraries. The polynucleotides of the invention may also be used as primers for the amplification of PD-ABC gene sequences encoding polynucleotides or a portion thereof through the PCR and other similar amplification procedures. The polynucleotides of the invention may also be used as probes and amplification primers to detect mutations in PD-ABC protein encoding genes that have been correlated with diseases, particularly diseases related to an altered function for PD-ABC proteins. Including, but not limited to, those diseases stated above.

The invention also provides a variety of polynucleotide expression vectors, comprising a PD-ABC gene, or a sequence substantially similar to it subcloned into an extra-chromosomal vector. This aspect of the invention allows for in vitro expression of the PD-ABC genes, thus permitting an analysis of PD-ABC gene regulation and PD-ABC protein structure and function. As used herein, the term “extra-chromosomal vector” includes, but is not limited to, plasmids, bacteriophages, cosmids, retroviruses and artificial chromosomes. In a preferred embodiment, the extra-chromosomal vector comprises an expression vector that allows for PD-ABC protein production when the recombinant DNA molecule is inserted into a host cell. Such vectors are well known in the art and include, but are not limited to, those with the T3 or T7 polymerase promoters, the SV40 promoter, the CMV promoter, or any promoter that either can direct gene expression, or that one wishes to test for the ability to direct gene expression.

In a preferred embodiment, the subject expression vectors comprise a polynucleotide sequence encoding an PD-ABC protein in functional combination with one or more promoter sequences so as to provide for the expression of the PD-ABC protein (or an anti-sense copy of the sequence suitable for inhibition of expression of an endogenous gene). The vectors may comprise additional polynucleotide sequences for gene expression, regulation, or the convenient manipulation of the vector, such additional sequences include terminators, reporters, enhancers, selective markers, packaging sites, and the like. Detailed description of polynucleotide expression vectors and their use can be found in, among other places Gene Expression Technology: Methods in Enzymology, Vol 185 Goeddel, ed, Academic Press Inc., San Diego, Calif. (1991), Protein Expression in Animal Cells, Roth, ed., Academic Press, San Diego, Calif. (1994).

The polynucleotide expression vectors of the invention have a variety of uses. Such uses include the genetic engineering of host cells to express PD-ABC proteins. In a further aspect, the present invention provides recombinant host cells that are stably transfected with a recombinant DNA molecule PD-ABC subcloned into an extra-chromosomal vector. The host cells of the present invention may be of any type, including, but not limited to, bacterial, yeast, mammalian cells, and Xenopus oocytes. Transfection of host cells with recombinant DNA molecules is well-known in the art (Sambrook, et al., Molecular Cloning, A Laboratory Manual, 2^{nd} ed., Cold Spring Harbor Press, 1989) and, as used herein, includes, but is not limited to calcium phosphate transfection, dextran sulfate transfection, electroporation, lipofection and viral infection. This aspect of the invention allows for in vitro and in vivo expression of PD-ABCs and their gene products, thus enabling high-level expression of PD-ABC proteins. In a further aspect of the invention the RNA molecules containing PD-ABCs can be injected into Xenopus oocytes and transport of substrates can be measured using standard electrophysiological techniques.

In another aspect of the invention transgenic animals can be constructed by injection of the nucloetide sequence for an PD-ABC cloned in suitable expression vectors into germ cells.

Other uses of the polynucleotide expression vectors, discussed in greater detail, infra, include, their use for genetic therapy for diseases and conditions in which it may be desirable use to express PD-ABC proteins at levels greater than naturally occurring expression levels. Alternatively, it may be desirable to use the subject vectors for antisense expression to reduce the naturally occurring levels of PD-ABC proteins.

The polynucleotide sequence of SEQ ID NOS 2 and 4 was mapped to human chromosomes using the nucleotide sequences for the cDNA from library sources to generate probes. The sequences were mapped to a particular chromosome or to a specific region of the chromosome using well-known techniques. These include in situ hybridization to chromosomal spreads, and PCR-based mapping by amplifying DNA from standard radiation hybrid cell lines. (Verma, et al., (1988) Human Chromosomes: A Manual of Basic Techniques, Pergamon Press, NYC).

In another aspect, the present invention provides a substantially purified recombinant protein comprising a polypeptide substantially similar to PD-ABC polypeptide shown in SEQ ID NOS 2 or 4. Furthermore, this aspect of the invention enables the use of PD-ABC proteins in several in vitro assays described below. As used herein, the term “substantially similar” includes deletions, substitutions and additions to the sequence of SEQ ID NOS 2 or 4 introduced by any in vitro means, or any genetic alterations naturally seen in vivo. As used herein, the term “substantially purified” means that the protein should be free from detectable contaminating protein, but the PD-ABC protein may be co-purified with an interacting protein, or as an oligomer. In a most preferred embodiment, the protein sequence according to the invention comprises an amino acid sequence of SEQ ID NOS 2 or 4. Mutated sequences according to the invention can be identified in a routine manner by those skilled in the art using the teachings provided herein and techniques well known in the art. This aspect of the invention provides a novel purified protein that can be used for in vitro assays, and as a component of a pharmaceutical composition.

PD-ABC proteins may be used to discover molecules that interfere with its activities. For example, molecules that prevent the binding of PD-ABC s to ligands or to other molecules.

The PD-ABC proteins of the present invention have a putative biological activity of modulating the cellular efflux of cholesterol. The PD-ABC proteins of the invention may be isolated from a variety of mammalian animal species. Preferred mammalian species for isolation are primates and humans. The invention also contemplates allelic variants of PD-ABC proteins may be prepared from a variety of mammalian tissues. Preferably PD-ABC proteins are obtained from recombinant host cells genetically engineered to express significant quantities of PD-ABC proteins. PD-ABC proteins may be isolated from non-recombinant or recombinant cells in a variety of ways well-known to a person of ordinary skill in the art.

The term “PD-ABC protein/s” as used herein refers not only to proteins having the amino acid residue sequence of naturally occurring PD-ABC proteins, but also refers to functional derivatives and variants of naturally occurring PD-ABC protein. A “functional derivative” of a native polypeptide is a compound having a qualitative biological activity in common with the native PD-ABC proteins. Thus, a functional derivative of a native PD-ABC protein is a compound that has a qualitative biological activity in common with a native PD-ABC protein, e.g., transporting substrates across biological membranes. “Functional derivatives” include, but are not limited to, fragments of native polypeptides from any animal species (including human), and derivatives of native (human and nonhuman) polypeptides and their fragments, provided that they have a biological activity in common with a respective native polypeptide. “Fragments” comprise regions within the sequence of a mature native polypeptide. The term “derivative” is used to define amino acid sequence and glycosylation variants, and covalent modifications of a native polypeptide, whereas the term “variant” refers to amino acid sequence and glycosylation variants within this definition. Preferably, the functional derivatives are polypeptides which have at least about 70% amino acid sequence similarity, more preferably about 80% amino acid sequence similarity, even more preferably at least 90% amino acid sequence similarity, most preferably at least about 99% amino acid sequence similarity with the sequence of a corresponding native polypeptide. Most preferably, the functional derivatives of a PD-ABC protein retain or mimic the region or regions within the native polypeptide sequence that directly participate in ligand binding. The phrase “functional derivative” specifically includes peptides and small organic molecules having a qualitative biological activity in common with a native PD-ABC protein.

“Identity” or “homology” with respect to a native polypeptide and its functional derivative is defined herein as the percentage of amino acid residues in the candidate sequence that are similar to residues of a corresponding native polypeptide, after aligning the sequences and introducing gaps, if necessary, to achieve the maximum percent homology. Neither N- or C-terminal extensions nor insertions, nor alternatively-spliced variants, shall be construed as reducing identity or homology. Methods and computer programs for the alignment are well-known in the art.

Amino acid sequence variants of native PD-ABC proteins or PD-ABC protein fragments are prepared by methods known in the art by introducing appropriate nucleotide changes into a native or variant PD-ABC proteins encoding DNA, or by in vitro synthesis of the desired polypeptides. There are two principal variables in the construction of amino acid sequence variants: the location of the mutation site and the nature of the mutation. With the exception of naturally-occurring alleles, which do not require the manipulation of the DNA sequence encoding the PD-ABC proteins, the amino acid sequence variants of PD-ABC proteins are preferably constructed by mutating the DNA, either to arrive at an allele or an amino acid sequence variant that does not occur in nature.

Alternatively or in addition, amino acid alterations can be made at sites that differ in PD-ABC proteins from various species, or in highly conserved regions, depending on the goal to be achieved.

Sites at such locations will typically be modified in series, e.g., by (1) substituting first with conservative choices and then with more radical selections depending upon the results achieved, (2) deleting the target residue or residues, or (3) inserting residues of the same or different class adjacent to the located site, or combinations of options 1 to 3.

One helpful technique is called “alanine scanning” Cunningham and Wells, Science, 1989;244:1081-1085. Here, a residue or group of target resides is identified and substituted by alanine or polyalanine. Those domains demonstrating functional sensitivity to the alanine substitutions are then refined by introducing further or other substituents at or for the sites of alanine substitution.

After identifying the desired mutation(s), the gene encoding a PD-ABC protein variant can, for example, be obtained by chemical synthesis.

More preferably, DNA encoding a PD-ABC protein amino acid sequence variant is prepared by site-directed mutagenesis of DNA that encodes an earlier prepared variant or a nonvariant version of the PD-ABC protein. Site-directed (site-specific) mutagenesis allows the production of PD-ABC protein variants through the use of specific oligonucleotide sequences that encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 20 to 25 nucleotides in length is preferred, with about 5 to 10 residues on both sides of the junction of the sequence being altered. In general, the techniques of site-specific mutagenesis are well-known in the art, as exemplified by publications such as, Edelman, et al., DNA, 1983;2:183. As will be appreciated, the site-specific mutagenesis technique typically employs a phage vector that exists in both a single-stranded and double-stranded form. Typical vectors useful in site-directed mutagenesis include vectors such as the M13 phage. This and other phage vectors are commercially available and their use is well-known to those skilled in the art. A versatile and efficient procedure for the construction of oligodeoxyribonucleotide directed site-specific mutations in DNA fragments using M13-derived vectors was published by Zoller, M. J. and Smith, M., Nucleic Acids Res., 1982;10:6487-6500. Also, plasmid vectors that contain a single-stranded phage origin of replication, Veira, et al., Meth. Enzymol., 1987;153:3 may be employed to obtain single-stranded DNA. Alternatively, nucleotide substitutions are introduced by synthesizing the appropriate DNA fragment in vitro, and amplifying it by PCR procedures known in the art.

In general, site-specific mutagenesis may be performed by obtaining either a double-stranded or a single-stranded vector that includes within its sequence a DNA sequence that encodes the relevant protein. An oligonucleotide primer bearing the desired mutated sequence is prepared, generally synthetically, for example, by the method of Crea, et al., Proc. Natl. Acad Sci. USA, 1978;75:5765. This primer is then annealed with the single-stranded protein sequence-containing vector, and subjected to DNA-polymerizing enzymes such as, E. coli polymerase I Klenow fragment, to complete the synthesis of the mutation-bearing strand. Thus, a heteroduplex is formed wherein one strand encodes the original nonmutated sequence and the second strand bears the desires mutation. This heteroduplex vector is then used to transform appropriate host cells such as HB101 cells, and clones are selected that include recombinant vectors bearing the mutated sequence arrangement. Thereafter, the mutated region may be removed and placed in an appropriate expression vector for protein production.

The PCR technique may also be used in creating amino acid sequence variants of a PD-ABC protein. When small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template. For introduction of a mutation into a plasmid DNA, one of the primers is designed to overlap the position of the mutation and to contain the mutation; the sequence of the other primer must be identical to a stretch of sequence of the opposite strand of the plasmid, but this sequence can be located anywhere along the plasmid DNA. It is preferred, however, that the sequence of the second primer is located within 500 to 5000 nucleotides from that of the first, such that in the end the entire amplified region of DNA bounded by the primes can be easily sequenced. PCR amplification using a primer pair like the one just described results in a population of DNA fragments that differ at the position of the mutation specified by the primer, and possibly at other positions, as template copying is somewhat error-prone.

Further details of the foregoing and similar mutagenesis techniques are found in general textbooks, such as, for example, Sambrook, et al., Molecular Cloning: H Laboratorv Manual 2nd edition, Cold Spring Harbor Press, Cold Spring Harbor (1989), and Current Protocols in Molecular Biology, Ausubel, et al. eds., John Wiley and Sons (1995).

Naturally-occurring amino acids are divided into groups based on common side chain properties:

(1) hydrophobic: norleucine, met, ala, val, leu, ile;

(2) neutral hydrophobic: cys, ser. tier;

(3) acidic: asp, glu;

(4) basic: asn, gin, his, lys, erg;

(5) residues that influence chain orientation: gly, pro; and

(6) aromatic: trp, tyr, pine.

Conservative substitutions involve exchanging a member within one group for another member within the same group, whereas non-conservative substitutions will entail exchanging a member of one of these classes for another. Variants obtained by nonconservative substitutions are expected to result in significant changes in the biological properties/function of the obtained variant, and may result in PD-ABC protein variants with PD-ABC protein biological activities, ie, modulation of cholesterol efflux. Amino acid positions that are conserved among various species are generally substituted in a relatively conservative manner if the goal is to retain biological function.

Amino acid sequence deletions generally range from about 1 to 30 residues, more preferably about 1 to 10 residues, and typically are contiguous. Deletions may be introduced into regions not directly involved in ligand binding.

Amino acid insertions include amino- and/or carboxyl terminal fusions ranging in length from one residue to polypeptides containing a hundred or more residues, as well as intrasequence insertions of single or multiple amino acid residues. Intrasequence insertions (i.e., insertions within the PD-ABC protein amino acid sequence) may range generally from about 1 to 10 residues, more preferably 1 to 5 residues, more preferably 1 to 3 residues. Examples of terminal insertions include the PD-ABC proteins with an N-terminal methionyl residue, a naturally-occurring N-terminal signal sequence, an artifact of direct expression in bacterial recombinant cell culture, and fusion of a heterologous N-terminal signal sequence to the N-terminus of the PD-ABC proteins to facilitate the secretion of mature PD-ABC proteins from recombinant host cells. Such signal sequences will generally be obtained from, and thus homologous to, the intended host cell species. Suitable sequences include STII or Ipp for E. coli, alpha factor for yeast, and viral signals such as herpes gD for mammalian cells. Other insertional variants of the native PD-ABC protein molecules include the fusion of the N- or C-terminus of an PD-ABC protein to immunogenic polypeptides, e.g., bacterial polypeptides such as betalactamase or an enzyme encoded by the E. cold trp locus, or yeast protein, and C-terminal fusions with proteins having a long half-life such as immunoglobulin regions (preferably immunoglobulin constant regions), albumin, or ferritin, as described in PCT published application WO 89/02922.

Since it is often difficult to predict in advance the characteristics of a variant PD-ABC protein, it will be appreciated that screening will be needed to select the optimum variant. For this purpose biochemical screening assays, such as those described herein below, will be readily available.

In a further aspect, the present invention provides antibodies and methods for detecting antibodies that selectively bind polypeptides with an amino acid sequence substantially similar to the amino acid sequence of SEQ ID NOS 2 or 4. As discussed in greater detail, infra, the antibody of the present invention can be a polyclonal or a monoclonal antibody, prepared by using all or part of the sequence of SEQ ID NOS 2 or 4, or modified portions thereof, to elicit an immune response in a host animal according to standard techniques (Harlow and Lane (1988), eds. Antibody: A Laboratory Manual, Cold Spring Harbor Press). In a preferred embodiment, the entire polypeptide sequence of SEQ ID NOS 2 or 4 is used to elicit the production of polyclonal antibodies in a host animal.

The method of detecting PD-ABC antibodies comprises contacting cells with an antibody that recognizes a PD-ABC protein and incubating the cells in a manner that allows for detection of the PD-ABC protein-antibody complex. Standard conditions for antibody detection of antigen can be used to accomplish this aspect of the invention (Harlow and Lane, 1988). This aspect of the invention permits the detection of PD-ABC proteins both in vitro and in vivo.

The subject invention provides methods for the treatment of a variety of diseases characterized by undesirably abnormal cellular levels of PD-ABCs. Diseases may be treated through either in vivo or in vitro genetic therapy. Protocols for genetic therapy through the use of viral vectors can be found, among other places, in Viral Vector Gene Therapy and Neuroscience Applications, Kaplit and Lowry, Academic Press, San Diego (1995). Gene therapy applications typically involve identifying target host cells or tissues in need of the therapy, designing vector constructs capable of expressing a desired gene product in the identified cells, and delivering the constructs to the cells in a manner that results in efficient transduction of the target cells. The cells or tissues targeted by gene therapy are typically those that are affected by the disease that the vector construct is designed to treat.

The genetic therapy methods of the present invention comprise the step of introducing a vector for the expression of a PD-ABC protein (or inhibitory antisense RNA) into a patient cell. The patient cell may be either in the patient, i.e., in vivo genetic therapy, or external to the patient and subsequently reintroduced into the patient, i.e., in vitro genetic therapy. Diseases that may be treated by the subject genetic therapy methods include, but are not limited to those associated with dyslipidemia Dyslipidemia, such as alterations in HDL metabolism, or CAD resulting from dyslipidemia has been associated with a number of diseases. Such diseases includes diabetes, fatty liver disease, obesity, insulin resistance, alcoholism, retinal degeneration, hypertension, and vascular diseases in general.

In a preferred aspect of the invention, a method is provided for protecting mammalian cells from abnormal levels of PD-ABCs in cells, comprising introducing into mammalian cells an expression vector comprising a DNA sequence substantially similar to the DNA sequence shown in SEQ ID NOS 1 or 3, that is operatively linked to a DNA sequence that promotes the expression of the DNA sequence and incubating the cells under conditions wherein the DNA sequence of SEQ ID NOS 1 or 3 will be expressed at high levels in the mammalian cells. Suitable expression vectors are as described above. In a preferred embodiment, the coding region of a human PD-ABC gene is subcloned into an expression vector under the transcriptional control of the cytomegalovirus (CMV) promoter to allow for constitutive PD-ABC gene expression.

In another preferred aspect of the present invention, a method is provided for treating or preventing abnormal levels of PD-ABC, comprising introducing into mammalian cells an expression vector comprising a DNA that is antisense to a sequence substantially similar to the DNA sequence shown in SEQ ID NOS 1 or 3 that is operatively linked to a DNA sequence that promotes the expression of the antisense DNA sequence. The cells are then grown under conditions wherein the antisense DNA sequence of SEQ ID NOS 1 or 3 will be expressed at high levels in the mammalian cells.

In a most preferred embodiment, the DNA sequence consists essentially of SEQ ID NOS 1 or 3. In a further preferred embodiment, the expression vector comprises an adenoviral vector wherein PD-ABC cDNA is operatively linked in an antisense orientation to a CMV promoter to allow for constitutive expression of the PD-ABC antisense cDNA in a host cell. In a preferred embodiment, the PD-ABC adenoviral expression vector is introduced into cells by injection into a mammal.

Another aspect of the invention is to provide assays useful for determining if a compound of interest can bind to PD-ABC proteins. This binding may interfere with, or mimic, the binding of ligands to the ABC1, or this binding may affect the function of PD-ABC in transporting substrates across membranes or modulating cholesterol efflux. The assay comprises the steps of measuring the binding of a compound of interest to a PD-ABC protein. Either the PD-ABC protein or the compound of interest to be assayed may be labeled with a detectable label, e.g., a radioactive or fluorescent label, so as to provide for the detection of complex formation between the compound of interest and the PD-ABC protein. In another embodiment of the subject assays, the assays involve measuring the interference, i.e., competitive binding, of a compound of interest with the binding interaction between PD-ABC proteins and a ligand already known to bind to ABC1 protein. For example, the effect of increasing quantities of a compound of interest on the formation of complexes between radioactivity labeled ligand and an PD-ABC protein may be measured by quantifying the formation of labeled ligand PD-ABC protein complex formation. In another embodiment of the subject assays, the assays involve measuring the alteration, ie, non-competitive inhibition, of a compound of interest with the activity of PD-ABC proteins.

Polyclonal antibodies to PD-ABC proteins generally are raised in animals by multiple subcutaneous (se) or intraperitoneal (ip) injections of a PD-ABC protein and an adjuvant. It may be useful to conjugate the PD-ABC protein or a fragment containing the target amino acid sequence to a protein that is immunogenic in the species to be immunized, eg, keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine resides), glutaraldehyde, succinic anhydride, SOCl₂, or R₁˜N═C═NR, where R and R₁ are different alkyl groups.

Animals are immunized against the immunogenic conjugates or derivatives by combining 1 mg or 1 fig of conjugate (for rabbits or mice, respectively) with 3 volumes of Freund's complete adjuvant and injecting the solution intradermally at multiple sites. One month later the animals are boosted with ⅕ to 1/10 the original amount of conjugate in Freund's complete adjuvant by subcutaneous injection at multiple sites. Seven to 14 days later the animals are bled and the serum is assayed for anti-PD-ABC protein antibody titer. Animals are boosted until the titer plateaus. Preferably, the animal is boosted with the conjugate of the same PD-ABC protein, but also may be conjugated to a different protein and/or through a different cross-linking reagent. Conjugates also can be made in recombinant cell culture as protein fusions. Also, aggregating agents such as alum are used to enhance the immune response.

Monoclonal antibodies are obtained from a population of substantially homogeneous antibodies, ie, the individual antibodies comprising the population are identical except for possible naturally-occurring mutations that may be present in minor amounts. Thus, the modifier “monoclonal” indicates the character of the antibody as not being a mixture of discrete antibodies. For example, the anti-PD-ABC protein monoclonal antibodies of the invention may be made using the hybridoma method first described by Kohler & Milstein, Nature, 1975;256:495, or may be made by recombinant DNA methods (Cabilly, et al, U.S. Pat. No. 4,816,567).

Antibodies can also be generated using phage display. In this approach libraries of peptides of random sequence are generated in antibody genes cloned into phage. These phage libraries are screened for antibodies by screening against the immobilized protein. (Hoogenboom-HR, Trends-Biotechnol. 1997; 15(2):62-70).

In the hybridoma method, a mouse or other appropriate host animal, such a hamster is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Coding, Monoclonal Antibodies: Principles and Practice, pp.59-103 [academic Press, 1986]).

The PD-ABC protein specific antibodies of the invention have a number of uses. The antibodies may be used to purify PD-ABC proteins from either recombinant or non-recombinant cells. The subject antibodies may be used to detect and/or quantify the presence of PD-ABC proteins in tissue samples, e.g., from blood, skin, and the like. Quantitation of PD-ABC proteins may be used diagnostically for those diseases and physiological or genetic conditions that have been correlated with particular levels of PD-ABC protein expression levels.

In a further aspect, the present invention provides a diagnostic assay for detecting cells containing PD-ABC deletions, comprising isolating total genomic DNA from the cell and subjecting the genomic DNA to PCR amplification using primers derived from the DNA sequence of SEQ ID NOS 1 or 3.

This aspect of the invention enables the detection of PD-ABC deletions in any type of cell, and can be used in genetic testing or as a laboratory tool. The PCR primers can be chosen in any manner that allows the amplification of an PD-ABC gene fragment large enough to be detected by gel electrophoresis. Detection can be by any method, including, but not limited to ethidium bromide staining of agarose or polyacrylamide gels, autoradiographic detection of radiolabeled PD-ABC gene fragments, Southern blot hybridization, and DNA sequence analysis. In a preferred embodiment, detection is accomplished by polyacrylamide gel electrophoresis, followed by DNA sequence analysis to verify the identity of the deletions. PCR conditions are routinely determined based on the length and base-content of the primers selected according to techniques well-known in the art (Sambrook, et al., 1989).

An additional aspect of the present invention provides a diagnostic assay for detecting cells containing PD-ABC deletions, comprising isolating total cell RNA and subjecting the RNA to reverse transcription-PCR amplification using primers derived from the DNA sequence of SEQ ID NOS 1 or 3. This aspect of the invention enables the detection of PD-ABC deletions in any type of cell, and can be used in genetic testing or as a laboratory tool.

Reverse transcription is routinely accomplished via standards techniques (Ausubel, et al., in Current Protocols in Molecular Biology, ed. John Wiley and Sons, Inc., 1994) and PCR is accomplished as described above.

The present invention may be better understood with reference to the accompanying examples that are intended for purposes of illustration only and should not be construed to limit the scope of the invention, as defined by the claims appended hereto.

Materials and Methods

Sequence Analyses

TBLASTN searches using ABC1 and ABCR protein sequences as queries were performed against a clustered EST database generated using the Compugen LEADS™ plarform. A single EST cluster consisting of four ESTs was identified (accession numbers AI733552, H21585). The 4 ESTs represent two different isoforms of PD-ABC. BLASTN searches of the high-throughput genomic sequence (HTG) division of the Genbank™ database led to the identification of an HTG sequence (accession number AC01558) which contains the complete coding region of PD-ABC.

Isolation of the cDNAs for PD-ABC

Two ESTs (I.M.A.G.E. #160038 and I.M.A.G.E. #182933) in the database contain partial open reading frames which share significant homology with human ABC1 after translation. The ESTs were obtained from ATCC and were sequenced completely. The ESTs have insert sizes of 1.2 kb (I.M.A.G.E. #160038) and 1.1 kb (I.M.A.G.E. #182933), respectively. The two clones are identical in their overlapping region of 1 kb. Using a region that is common to both ESTs as probe, cDNA clones were isolated from a mixture of three human cDNA libraries: adult brain, skeletal muscle, and mammary gland (EdgeBiosystems, Gaithersburg, Md.). DNA sequencing was carried out with universal and synthetic primers. PCR amplification of the 5′ cDNA ends

The longest clone was sequenced and was found to be missing the 5′ end. Database searches generated high throughput genomic sequence clusters that share identity with the cDNA sequence we obtained. Three pairs of primers were synthesized based on the high throughput genomic sequences. These primer pairs areas follows:

| | | |
|---|---|---|
| 1. | forward primer, | 5′-TCTCACCATGGCCTTCTGGACACAG-3′ |
| | reverse primer, | 5′-CACGTAGCGCAGGTCGGTCAGGG-3′ |
| 2. | forward primer, | 5′-GCTGATTGGAGCCCTGGACAGCCA-3′ |
| | reverse primer, | 5′-GTCCACATAGCACGGATAGGGCAT-3′ |
| 3. | forward primer, | 5′-TCGTGTACCTGCAAGACCTGGTG-3′ |
| | reverse primer, | 5′-CAGAGCCAGGCTCTCGCAGCC-3′ |

PCR reactions were carried out with human pituitary gland or thymus Marathon-Ready cDNAs (Clontech). The reaction was started with an initial denaturation of 5 minutes at 94° C., followed by 28 cycles of 30 seconds at 94° C., 2 minutes at 60° C., 2 minutes at 72° C. with a final extension of 10 minutes at 72° C. PCR reactions with the three pairs of primers generated three bands with the following sizes, respectively: 1.8 kb, 800 bp, and 600 bp. The PCR products were ligated into pCRII-TOPO vector (Invitrogen) and sequenced with universal and synthetic primers.

Northern Blotting Analysis

A digoxigenin (DIG)-labeled probe for Northern blotting was generated using a PCR labeling kit (Boehringer Mannheim) with primers based on the sequence of a partial PD-ABC cDNA fragment. The forward primer was 5′-CAGCTTCACTCTTGTCCTCATTGAG-3′ and the reverse primer was 5′-TTTATGCAGGTGAGCACCACATAG-3′. The 262 bp PCR product was gel-purified and used for Northern blotting. The template for the PCR was either PD-ABC partial cDNA fragment or human spleen cDNA (Clontech). The 12-tissue master blot (Origene, Rockville, Md.) and a 6-tissue master blot (Clontech) were hybridized with the probe and developed according to the manufacturer's instruction (Boehringer Mannheim). The same blots were stripped and hybridized with a DIG-labeled GAPDH or β-actin probe for control purpose.

Tissue Distribution by RT-PCR

The tissue-specific expression of the two PD-ABC variants was carried out by reverse transcription-polymerase chain reaction (hereinafter “RT-PCR”). Rapid-Scan Gene Expression Panels (Origene) were used as PCR templates. Primers specific for Form 1 are 5′-CCCCTCTTCCTTCTCTTCACACTAC-3′ (forward primer) and 5′-AGCAGCCCAAAACACTCACCAC-3′ (reverse primer); primers specific for Form 2 are 5′-TGGGAGAGGAGGACGAGGATGTAG-3′ (forward primer) and 5′-AGGTGTTCAGTAAAGGATGATGGG-3′ (reverse primer). The PCR reaction was carried out with 35 cycles as follows: 95° C., 1 minute; 62° C., 1 minute; 72° C., 1 minute. The PCR products were separated on 1% NuSieve gels (FMC).

Results and Discussion

Isolation and Primary Structure of PD-ABC

ABC1 is a member of ABCA subfamily (Broccardo, C. et al., Biochim. Biophys. Acta, 1999; 1461:395-404) and is associated with TD. Recent pharmacological studies showed that ABC1 is responsible for cholesterol and phospholipid transport (Lawn, R. M., et al., J. Clin. Invest. 104, R25-31, 1999.). Two additional ABCA subfamily members, ABC2 and ABCR, have been described (Luciani, M -F., et al., EMBO J., 1996;15:226-235; Allikmets, R., et al., Nat. Genet, 1997;15:236-246). The functions of ABCR and ABC2 are unknown though ABCR has been proposed as a flipase for N-retinylidene-phosphatidylethanolamine (Weng, J., et al. Cell, 1999;98:13-23). To search for other ABC1 homologues, especially those that are also involved in cholesterol metabolism, we searched the database for novel sequences that share homology with ABC1 and ABCR. Two overlapping ESTs were identified which contain a partial open reading frame. The 5′ end 80% of the open reading frame is similar with ABC1. While the 3′ end 20% of the open reading frame does not share any homology with ABC1. In addition, this open reading frame does not contain the corresponding NBF as predicted in ABC1 (Bodzioch, M., et al., Nature Genetics, 1999;22:347-351; Brooks-Wilson, A., et al., Nature Genetics, 1999;22:336-345; Rust, S., et al., Nature Genetics, 1999;22:352-355).

Using a probe based on the sequence homologous to ABC1, we obtained cDNA clones from human cDNA libraries. The longest clone was sequenced and it is identical to the EST sequence in the 5′ end. Interestingly, the 3′ end of this clone is different from that of the EST. In addition, this clone contains the second NBF after translation and the entire amino acid sequence share homology with ABC1. We predicted that there are two forms of PD-ABC originating from alternative splicing. The cDNA clone represents Form 1, which contains the second NBF; the EST represents Form 2, which lacks the second NBF. We identified the alternative splice site shown in FIG. 2 and the sizes of introns and exons as well as the intron-exon bound areas are shown below in Tables 1 and 2.

TABLE 1

| Exon No | Exon Size | Intron Size | 5′ Splice Site | | 3′ Splice Site | | Position, bp |
|---|---|---|---|---|---|---|---|
| 1 | 67 | | | | gacagccggt | AACGCCCCAG | 68 |
| 2 | 95 | 80 | CTCCCCGCAG | ggtccagctc | accatgaatg | GTGAGCCAGA | 161 |
| 3 | 142 | 227 | TGGCTTGTTT | gccacttccc | cgactccctg | TGAGCCAGAG | 303 |
| 4 | 112 | 91 | CTGGGGACAG | ggtctcccgg | cgcagcacgg | GTGAGGAGGC | 415 |
| 5 | 84 | 138 | TCCTCCCCCA | gcccagcctc | ctgcgcacgg | TAGGGTGTCG | 499 |
| 6 | 81 | 347 | TTTCTCCCCA | ggaatccctg | ggcccaggag | GTACGAGGCC | 579 |
| 7 | 213 | 213 | ACCTCTCTCT | agctcctggc | gcagcctgag | TGAGTGACTG | 791 |
| 8 | 142 | 82 | CCCATCCCAG | gccccgcctg | ggcccaggtg | GGGGCAGCCT | 933 |
| 9 | 119 | 246 | GTGCTGTCCA | caggtgaacc | catgctgcag | CCCCGCACAC | 1047 |
| 10 | 170 | 733 | CTGCGCCCCC | cagcggctcc | agtgacggag | AGGCCCTCAC | 1215 |
| 11 | 233 | 256 | GTGGGATGCG | agtgcctgtc | agggacaggt | CAGGCGAGGG | 1447 |
| 12 | 181 | 994 | GCCCCTCTCG | caggttttgg | gacgacgtgt | GCCAGAGCTC | 1624 |
| 13 | 227 | 394 | CCCACCCCAG | gttcctgcgt | tcaagctggg | CGCGCCTCGG | 1850 |
| 14 | 223 | 126 | TGCCTCTCAC | agctggggga | cgtggccgcg | GTGAGAGCCG | 2067 |
| 15 | 203 | 74 | CTTTTCCGCA | gagcctgctg | tgtgcccagg | TGGGCCGATG | 2270 |
| 16 | 113 | 1237 | CCCCTCCCCG | caggccagta | gacccaaagg | GTGAGGCACT | 2380 |
| 17 | 174 | 260 | CATCTCTGCA | gtgctggtag | accaccctgt | GAGCCCCCAA | 2554 |
| 18 | 132 | undet. | CTATCCACAG | gtccatcttg | gtttgacatg | TGCGTCTCGG | 2685 |
| 19 | 142 | 102 | CTGGCCGCAG | gctgaccgtg | ctctctggtg | AGCCCATCCC | 2827 |
| 20 | 140 | 151 | ATCTCTACCA | ggtgggatgc | ccgagaaggt | AAGAGCTGGG | 2964 |
| 21 | 187 | 352 | CCCCGTGCCT | aggtcgcacg | aatgagaagg | TGGGGACCGG | 3148 |
| 22 | 75 | 85 | CTCTGCCCGC | aggctgacac | gcagagtcgg | TGAGGGCCGG | 3221 |
| 23 | 203 | undet. | TGTCCACACA | ggcactcctc | cctcgaggag | GTGTGAGGCC | 3423 |
| 24 | 50 | 255 | GTCTCCCCAG | atcttcctga | gatatggagg | GTGCGGCCAC | 3472 |
| 25 | 105 | 169 | TGCAGGGCCA | gatggcagct | ggggaaccag | GTAAGTCCTT | 3577 |
| 26 | 151 | 80 | TCATCCCTCA | cagctgggtc | gttcgcccag | GTGAGGAGGG | 3726 |
| 27 | 129 | 224 | CTGCACCCAC | cccagatcgt | ccttcttcag | GTGGGTGCAG | 3851 |
| 28 | 103 | 83 | CCCTCACACA | cagtgaggac | tcccacaggt | GAGGCGTCTT | 3952 |
| 29 | 257 | 214 | CCATTGTCTG | caggttctcg | tgcgccaggg | GTGAGCCATG | 4205 |
| 30 | 35 | 555 | CTCTCCCACA | gcctgaagac | gaggtcaggt | GAGGAGGGGT | 4240 |
| 31 | 176 | 126 | GCCCCACAGA | tacggaggct | cagtctcaag | GTGGGAACTG | 4416 |
| 32 | 172 | 85 | ACCCCCACCC | agatctggtt | ggctgcactg | GAGGGACTCA | 4587 |
| 33 | 177 | 407 | TCACCTCCAG | gatggcctcc | ctgggacatg | TGCGGGGGCG | 4764 |
| 34 | 116 | 229 | CTCCAATGCA | gtgtaactac | tactgtatgg | GTGAGGCCCC | 4880 |
| 35 | 145 | 485 | GCCTGTGCCC | gctggtcgat | ctctgatcag | GTGGGGCACC | 5025 |
| 36 | 127 | 84 | CACCCTTGAG | cagaagctgc | agcgcttggg | GAGGCGGAGG | 5150 |
| 37 | 131 | 347 | TGTGGATATA | ggagacaggc | cctgccacag | TTAGTGAGGT | 5280 |
| 38 | 123 | 72 | CTGTCCCCAG | gcccagggtg | gaccaaggta | GGTGTGGTCA | 5403 |
| 39 | 64 | 78 | GCACTCTCCC | aggtataccg | ccctggtgag | GTGAGTCCAG | 5463 |
| 40 | 108 | 2695 | GCATCCCTGT | agtgttttgg | caggccacag | GTGAGGGGTG | 5570 |
| 41 | 144 | 281 | TGTCCCTTAT | cagcgtggcc | ggttgcccag | GTGAGCCCAC | 5712 |
| 42 | 138 | 1228 | CCCCACCCCA | cagaccgctg | gtgtttctgg | TGCGTGGGAG | 5848 |
| 43 | 104 | 80 | GTGCTCCCCA | ggacgagccg | cctcccatag | GTGGGCCGGG | 5951 |
| 44 | 94 | 296 | GGGTCCCGAC | agcatggagg | tcaagggcag | GTGAGCCGGC | 6044 |
| 45 | 246 | 674 | CCCACTCACT | gcagattcgc | tggaggaggt | GATCACGGCG | 6287 |
| 46 | 232 | 95 | GCCCACCGCT | aggtattctt | tangagccct | GGACTCAGGC | 6516 |

TABLE 2

| Exon No. | Exon Size | Intron Size | 5′ Splice Site | | 3′ Splice Site | | Position, bp |
|---|---|---|---|---|---|---|---|
| 1 | 67 | | | | gacagccggt | AACGCCCCAG | 68 |
| 2 | 95 | 80 | CTCCCCGCAG | ggtccagctc | accatgaatg | GTGAGCCAGA | 161 |
| 3 | 142 | 227 | TGGCTTGTTT | gccacttccc | cgactccctg | TGAGCCAGAG | 303 |
| 4 | 112 | 91 | CTGGGGACAG | ggtctcccgg | cgcagcacgg | GTGAGGAGGC | 415 |
| 5 | 84 | 138 | TCCTCCCCCA | gcccagcctc | ctgcgcacgg | TAGGGTGTCG | 499 |
| 6 | 81 | 347 | TTTCTCCCCA | ggaatccctg | ggcccaggag | GTACGAGGCC | 579 |
| 7 | 213 | 213 | ACCTCTCTCT | agctcctggc | gcagcctgag | TGAGTGACTG | 791 |
| 8 | 142 | 82 | CCCATCCCAG | gccccgcctg | ggcccaggtg | GGGGCAGCCT | 933 |
| 9 | 119 | 246 | GTGCTGTCCA | caggtgaacc | catgctgcag | CCCCGCACAC | 1047 |
| 10 | 170 | 733 | CTGCGCCCCC | cagcggctcc | agtgacggag | AGGCCCTCAC | 1215 |
| 11 | 233 | 256 | GTGGGATGCG | agtgcctgtc | agggacaggt | CAGGCGAGGG | 1447 |
| 12 | 181 | 994 | GCCCCTCTCG | caggttttgg | gacgacgtgt | GCCAGAGCTC | 1624 |
| 13 | 227 | 394 | CCCACCCCAG | gttcctgcgt | tcaagctggg | CGCGCCTCGG | 1850 |
| 14 | 223 | 126 | TGCCTCTCAC | agctggggga | cgtggccgcg | GTGAGAGCCG | 2067 |
| 15 | 203 | 74 | CTTTTCCGCA | gagcctgctg | tgtgcccagg | TGGGCCGATG | 2270 |
| 16 | 113 | 1237 | CCCCTCCCCG | caggccagta | gacccaaagg | GTGAGGCACT | 2380 |
| 17 | 174 | 260 | CATCTCTGCA | gtgctggtag | accaccctgt | GAGCCCCCAA | 2554 |
| 18 | 132 | undet. | CTATCCACAG | gtccatcttg | gtttgacatg | TGCGTCTCGG | 2685 |
| 19 | 142 | 102 | CTGGCCGCAG | gctgaccgtg | ctctctggtg | AGCCCATCCC | 2827 |
| 20 | 140 | 151 | ATCTCTACCA | ggtgggatgc | ccgagaaggt | AAGAGCTGGG | 2964 |
| 21 | 187 | 352 | CCCCGTGCCT | aggtcgcacg | aatgagaagg | TGGGGACCGG | 3148 |
| 22 | 75 | 85 | CTCTGCCCGC | aggctgacac | gcagagtcgg | TGAGGGCCGG | 3221 |
| 23 | 203 | undet. | TGTCCACACA | ggcactcctc | cctcgaggag | GTGTGAGGCC | 3423 |
| 24 | 50 | 255 | GTCTCCCCAG | atcttcctga | gatatggagg | GTGCGGCCAC | 3472 |
| 25 | 105 | 169 | TGCAGGGCCA | gatggcagct | ggggaaccag | GTAAGTCCTT | 3577 |
| 26 | 151 | 80 | TCATCCCTCA | cagctgggtc | gttcgcccag | GTGAGGAGGG | 3726 |
| 27 | 129 | 224 | CTGCACCCAC | cccagatcgt | ccttcttcag | GTGGGTGCAG | 3851 |
| 28 | 103 | 83 | CCCTCACACA | cagtgaggac | tcccacaggt | GAGGCGTCTT | 3952 |
| 29 | 257 | 214 | CCATTGTCTG | caggttctcg | tgcgccaggg | GTGAGCCATG | 4205 |
| 30 | 35 | 555 | CTCTCCCACA | gcctgaagac | gaggtcaggt | GAGGAGGGGT | 4240 |
| 31 | 176 | 126 | GCCCCACAGA | tacggaggct | cagtctcaag | GTGGGAACTG | 4416 |
| 32 | 172 | 85 | ACCCCCACCC | agatctggtt | ggctgcactg | GAGGGACTCA | 4587 |
| 33 | 177 | 407 | TCACCTCCAG | gatggcctcc | ctgggacatg | TGCGGGGGCG | 4764 |
| 34 | 116 | 229 | CTCCAATGCA | gtgtaactac | tactgtatgg | GTGAGGCCCC | 4880 |
| 35 | 145 | 485 | GCCTGTGCCC | gctggtcgat | ctctgatcag | GTGGGGCACC | 5025 |
| 36 | 127 | 84 | CACCCTTGAG | cagaagctgc | agcgcttggg | GAGGCGGAGG | 5150 |
| 37 | 131 | 347 | TGTGGATATA | ggagacaggc | cctgccacag | TTAGTGAGGT | 5280 |
| 38a | 123 | 72 | CTGTCCCCAG | gcccagggtg | | | |

Using a variety of techniques (Materials and Methods), we obtained the full length PD-ABC coding region. The full length PD-ABC contains an open reading frame of 2146 amino acids and is a typical ABC transporter. PD-ABC is currently the closest ortholog to ABC1 in the public database. The sequence was aligned with ABC1 and ABCR (FIG. 1). The homology between PD-ABC and ABC1 is 66%. The most conserved regions of the PD-ABC alignment corresponds to the transmembrane and nucleotide binding domains.

The existence of two splice variants of PD-ABC is interesting, especially that Form 2 does not contain the second NBF (FIG. 2). In ABC transporters, the NBFs are required for ATP binding and hydrolysis, which provides energy for transport of substrates. Lack of the second NBF in other ABC transporters usually results in a dysfunctional transporter. In certain TD patients, nonsense mutations or deletions at the C-terminus of ABC1 are responsible for the loss of cholesterol efflux, suggesting that the second NBF is required for transporter activity. Loss of the second NBF in Form 2, PD-ABC may result in a transporter that does not have any activity. This transporter, however, may still be capable of binding substrates and therefore, serves as a regulator of transport by competing for substrates with Form 1 transporter. Alternatively, the first NBF might be essential to provide energy for transport as in the half-size ABC transporters like ABC8 (Klucken, J., et al., Proc. Natl. Acad. Sci. USA, 2000;97:817-822).

Predicted Genetic Structure of PD-ABC

The overall structure of the PD-ABC is outlined in FIG. 2. Using the full length cDNA sequence, we identified two overlapping genomic sequences in the high throughput genomic database which align to the complete PD-ABC coding sequence. No stop codons were found in the PD-ABC genomic sequence, indicating this is not a pseudogene. The genomic sequences for PD-ABC are both derived from human chromosome 19p13.3.

By aligning the cDNA sequence of PD-ABC to the genomic sequences, we were able to determine the intron-exon boundaries of the gene (FIG. 2B). The coding region of Form 1 of PD-ABC is contained in 47 exons, and covers 20 kb of genomic sequence (FIG. 2). Form 2 of PD-ABC utilizes an alternative polyadenylation signal found in intron number 38. This results in a truncation of the PD-ABC transcript. The intron/exon boundaries of PD-ABC Interestingly, the intron/exon structure of PD-ABC is highly similar to that of ABC1 and ABCR (data not shown).

Tissue Distribution of PD-ABC

The tissue distribution of PD-ABC was examined by Northern blotting analysis using a probe common to both Form 1 and Form 2. A band with a size between 8 and 9 kb was observed (FIG. 3A). The transcript was only detected in spleen, suggesting that PD-ABC is specifically expressed in spleen. No expression was observed in the other tissues examined, including brain, heart, lung, liver and muscle. The same blot was further hybridized with a GAPDH probe to show that the spleen-specific expression is not a result of unequal loading of mRNA samples.

The spleen-specific expression prompted us to examine the expression of PD-ABC in immune system cells or tissues. Indeed, we found PD-ABC is highly expressed in the immune system tissues tested, including lymph node, thymus, peripheral blood leukocytes, bone marrow and fetal liver (FIG. 3B). Interestingly, there are two bands in peripheral blood leukocytes and fetal liver. The two transcripts are almost equally expressed in both tissues, while the smaller message is only mildly expressed in bone marrow. The two transcripts may represent the two variants we identified (FIG. 3A). To further assess PD-ABC expression in various tissues in a broader scope, we carried out dot blot analysis with human tissues (Table 3).

TABLE 3
Expression of PD-ABC in various human tissues assessed by dot blot
analysis

| | Relative PD-ABC1 | | Relative PD-ABC1 |
|---|---|---|---|
| Tissue | mRNA expression | | mRNA expression |
| Whole Brain | * | Colon, transverse | * |
| Cerebral cortex | * | Colon, descending | * |
| Frontal lobe | * | Rectum | * |
| Parietal lobe | * | Kidney | * |
| Parietal lobe | * | Skeletal muscle | * |
| Temporal lobe | * | Spleen | *** |
| P.g* of cerebral cortex | * | Thymus | **** |
| Pons | * | Peripheral blood leukocyte | **** |
| Cerebellum left | * | Lymph node | **** |
| cerebellum right | * | Bone marrow | **** |
| Corpus callosum | * | Trachea | * |
| Amydala | * | Lung | * |
| Caudate nucleus | * | Placenta | * |
| Hippocampus | * | Bladder | * |
| Medulla oblongata | * | Uterus | * |
| Putamen | * | Prostate | * |
| Substantia nigra | * | Testis | * |
| Accumbens nucleus | * | Ovary | * |
| Thalamus | * | Liver | * |
| Pituitary gland | **** | Pancreas | * |
| Spinal cord | * | Adrenal gland | * |
| Heart | * | Mammary gland | * |
| Aorta | * | Leukemia HL-60 | ** |
| Atrium right | * | Hela S3 | ** |
| Atrium left | * | Leukemia, k-562 | ** |
| Ventrical left | * | leukemia, MOLT-4 | * |
| Ventrical right | * | Burkitt's lymphoma | * |
| Interventricular septum | * | Colorectal adenacarcinoma | * |
| Apex of the heart | * | Lung carcinoma | * |
| Esophagus | * | Fetal brain | * |
| Stomach | * | Fetal heart | * |
| Duodenum | * | Fetal kidney | * |
| Jejunum | * | Fetal liver | *** |
| Ileum | * | Fetal spleen | *** |
| Ilocecum | * | Fetal thymus | *** |
| Appendix | * | Fetal lung | * |
| | | Colon, ascending | * |

The dot blot was quantitated densitometrically and the values were expressed as number of dots on a linear scale.

Consistent with that in FIG. 3B, PD-ABC is primarily expressed in the immune system. In addition, it is also highly expressed in the pituitary gland.

The expression pattern of PD-ABC in the immune system suggests that PD-ABC may have a physiological role in those tissues or organs. The link of ABC transporters to the immune system has been documented previously. ABC1 is required for engulfment of apoptotic cells by macrophages. In addition, there is a close correlation between interleukin-1β secretion and ABC1 activity as demonstrated in studies with ABC1 inhibitors (Harmon, Y., et al., Blood, 1997;90:2911-2915). These findings suggest that ABC1 might be involved in interleukin-1β secretion and play roles in inflammatory reactions. Given the great homology between PD-ABC and ABC1, the two transporters may have similar biological functions. In contrast to the ubiquitous expression of ABC1, PD-ABC expression is almost immune system-specific. This is a strong indication that PD-ABC involved in certain immunological pathways.

Expression of the Two Isoforms of PD-ABC

The expression of the two variants was examined in tissues with RT-PCR. Although RT-PCR is not absolutely quantitative, the appearance of the PCR product in templates from different tissues can provide a general trend of transcript abundance. We examined the expression of the two PD-ABC variants in 24 human tissues and found that the expression patterns are different. Most of the 24 tissues express both Form 1 and Form 2 (FIG. 4). However, prostate and ovary preferentially express Form 1. While tissues including fetal brain, skin, uterus, pancreas, adrenal gland, salivary gland and colon preferentially express Form 2 (FIG. 4). Interestingly, we found a larger band in bone marrow and peripheral blood leukocytes with the primers specific to Form 2 (FIG. 4). This suggests that there might be an another form of PD-ABC without the second NBF in these tissues. The lack of the second NBF in Form 2 most likely affects the transporter activity and the tissue-specific expression of this form might serve a special physiological purpose.

Conclusion

A new ABC transporter, which is the closest ABC1 homologue, has been identified and isolated. In addition, an alternatively spliced variant was identified. The transporter is primarily expressed in the immune system and may play a role in immune responses. Further, the expression of the smaller, alternatively spliced transcript of PD-ABC is more restricted than the original form. The tissue-specific expression pattern and alternative splicing of PD-ABC suggest that PD-ABC might have a similar function as ABC1, but in a more restricted and regulated manner.

It is to be understood that the invention is not to be limited to the exact details of operation, or to the exact compounds, compositions, methods, procedures or embodiments shown and described, as obvious modifications and equivalents will be apparent to one skilled in the art, and the invention is therefore to be limited only by the full scope of the appended claims.

We claim:

1. An isolated and purified DNA sequence substantially similar to the DNA sequence shown in SEQ ID NOS 1 or 3.

2. An isolated and purified DNA sequence that hybridizes to the DNA sequence shown in SEQ ID NOS 1 or 3 under high stringency hybridization conditions.

3. An isolated and purified DNA sequence that consists essentially of the DNA sequence shown in SEQ ID NOS 1 or 3.

4. An isolated and purified DNA sequence that has at least a 70% identity to a polynucleotide encoding the polypeptide expressed by SEQ ID NOS 2 or 4.

5. An isolated and purified DNA sequence that is fully complementary to the DNA sequence shown in SEQ ID NOS 1 or 3.

6. A recombinant DNA molecule comprising the isolated and purified DNA sequence of claim 2 or 3 subcloned into an extra-chromosomal vector.

7. A recombinant host cell comprising a host cell transfected with the recombinant DNA molecule of claim 6.

8. A substantially purified recombinant polypeptide, wherein the amino acid sequence of the substantially purified recombinant polypeptide is substantially similar to the amino acid sequence shown in SEQ ID NOS 2 or 4.

9. A substantially purified recombinant polypeptide of claim 8, wherein the polypeptide has at least about 70% amino acid sequence similarity to the amino acid sequence shown in SEQ ID NOS 2 or 4.

10. A substantially purified recombinant polypeptide, wherein the amino acid sequence of the substantially purified recombinant polypeptide consists essentially of the amino acid sequence shown in SEQ ID NOS 2 or 4.

11. An antibody that selectively binds polypeptides with an amino acid sequence substantially similar to the amino acid sequence of claim 8.

12. A method of detecting PD-ABC protein in cells, comprising contacting cells with the antibody of claim 11 and incubating the cells in a manner that allows for detection of the PD-ABC protein-antibody complex.

13. A diagnostic assay for detecting cells containing PD-ABC mutations, comprising isolating total genomic DNA from the cell and subjecting the genomic DNA to PCR amplification using primers derived from the isolated and purified DNA sequence of claim 1, 2, or 3 or by analyzing the genomic DNA directly by a hybridization method and determining whether the resulting PCR product contains a mutation.

14. A diagnostic assay for detecting cells containing PD-ABC mutations, comprising isolating total cell RNA, subjecting the RNA to reverse transcription-PCR amplification using primers derived from the isolated and purified DNA sequence of claim 1, 2, or 3 and determining whether the resulting PCR product contains a mutation.

15. A method for the amplification of a region of the DNA sequence of claim 1, 2, or 3, the method comprising the step of: contacting a test sample suspected of containing the desired sequence of claim 1, 2, or 3 or portion thereof with amplification reaction reagents.

16. A diagnostic kit for detecting the presence of at least one copy of the DNA sequence of claim 1, 2, or 3 in a test sample, said kits containing a primer, a pair of primers or a probe, and optionally amplification reagents.

17. An assay for the detection or screening of therapeutic compounds that interfere with or mimic the interaction between the polypeptide of claim 8, 9, or 10 and ligands that bind to the polypeptide of claim 8, 9, or 10.

18. The assay of claim 17, herein the assay comprises the steps of:
a) providing a polypeptide of claim 8, 9, or 10;
b) obtaining a candidate substance;
c) bringing into contact said polypeptide with said candidate substance; and
d) detecting the complexes formed between said polypeptide and said candidate substance.

19. A method for protecting mammalian cells from abnormal calcium flux, comprising introducing into mammalian cells an expression vector comprising the isolated and purified DNA sequence of claim 1, 2, or 3, which is operatively linked to a DNA sequence that promotes the high level expression of the isolated and purified DNA sequence in mammalian cells.

20. A method for treating or preventing epilepsy, comprising introducing into a mammal an expression vector comprising the isolated and purified DNA sequence of claim 1, 2, or 3, which is operatively linked to a DNA sequence that promotes the high level expression of the antisense strand of the isolated and purified DNA sequence in mammalian cells.

21. A method for purifying PD-ABC protein from cells, comprising:
transfecting a host cell with a vector comprising the isolated and purified DNA sequence of claim 1, 2, or 3 operatively linked to a promoter capable of directing gene expression in a host cell;
inducing expression of the isolated and purified DNA sequence in the cells;
lysing the cells;
isolating PD-ABC protein from the cells ; and
purifying PD-ABC protein from the isolate.

22. A method treating or preventing a disease selected from the group comprised of dyslipidemia-related syndromes wherein the method comprises the step of administering the polynucleotide of claims 1, 2, or 3 to a mammal in need thereof a therapeutically effective amount.

23. A method treating or preventing a disease selected from the group comprised of dyslipidemia-related syndromes wherein the method comprises the step of administering the polypeptide of claims 8, 9, or 10 to a mammal in need thereof a therapeutically effective amount.


SEQUENCE LISTING (18 sequences)

SEQ ID NO: 1 - DNA, 6522 bp, Homo sapiens
  unsure (6509): Any
atggccttctggacacagctgatgctgctgctctggaagaatttcatgtatcgccggagacagccggtccagctcctggtcgaattgctgtggcctctcttcctcttcttcatcctggtggctgttcgccactcccacccgcccctggagcaccatgaatgccacttcccaaacaagccactgccatcggcgggcaccgtgccctggctccagggtctcatctgtaatgtgaacaacacctgctttccgcagctgacaccgggcgaggagcccgggcgcctgagcaacttcaacgactccctggtctcccggctgctagccgatgcccgcactgtgctgggaggggccagtgcccacaggacgctggctggcctagggaagctgatcgccacgctgagggctgcacgcagcacggcccagcctcaaccaaccaagcagtctccactggaaccacccatgctggatgtcgcggagctgctgacgtcactgctgcgcacggaatccctggggttggcactgggccaagcccaggagcccttgcacagcttgttggaggccgctgaggacctggcccaggagctcctggcgctgcgcagcctggtggagcttcgggcactgctgcagagaccccgagggaccagcggccccctggagttgctgtcagaggccctctgcagtgtcaggggacctagcagcacagtgggcccctccctcaactggtacgaggctagtgacctgatggagctggtggggcaggagccagaatccgccctgccagacagcagcctgagccccgcctgctcggagctgattggagccctggacagccacccgctgtcccgcctgctctggagacgcctgaagcctctgatcctcgggaagctactctttgcaccagatacaccttttacccggaagctcatggcccaggtgaaccggaccttcgaggagctcaccctgctgagggatgtccgggaggtgtgggagatgctgggaccccggatcttcaccttcatgaacgacagttccaatgtggccatgctgcagcggctcctgcagatgcaggatgaaggaagaaggcagcccagacctggaggccgggaccacatggaggccctgcgatcctttctggaccctgggagcggtggctacagctggcaggacgcacacgctgatgtggggcacctggtgggcacgctgggccgagtgacggagtgcctgtccttggacaagctggaggcggcaccctcagaggcagccctggtgtcgcgggccctgcaactgctcgcggaacatcgattctgggccggcgtcgtcttcttgggacctgaggactcttcagaccccacagagcacccaaccccagacctgggccccggccacgtgcgcatcaaaatccgcatggacattgacgtggtcacgaggaccaataagatcagggacaggttttgggaccctggcccagccgcggaccccctgaccgacctgcgctacgtgtggggcggcttcgtgtacctgcaagacctggtggagcgtgcagccgtccgcgtgctcagcggcgccaacccccgggccggcctctacctgcagcagatgccctatccgtgctatgtggacgacgtgttcctgcgtgtgctgagccggtcgctgccgctcttcctgacgctggcctggatctactccgtgacactgacagtgaaggccgtggtgcgggagaaggagacgcggctgcgggacaccatgcgcgccgtggggctcagccgcgcggtgctctggctaggctggttcctcagctgcctcgggcccttcctgctcagcgccgcgctgctggttctggtgctcaagctgggggacatcctcccctgcagccacccgggcgtggtcttcctgttcttggcagccttcgcggtggccacggtgacccagagcttcctgctcagcgccttcttctcccgcgccaacctggctgcggcctgcggcggcctggcctacttctccctctacctgccctacgtgctgtgtgtggcttggcgggaccggctgcccgcgggtggccgcgtggccgcgagcctgctgtcgcccgtggccttcggcttcggctgcgagagcctggctctgctggaggagcagggcgagggcgcgcagtggcacaacgtgggcacccggcctacggcagacgtcttcagcctggcccaggtctctggccttctgctgctggacgcggcgctctacggcctcgccacctggtacctggaagctgtgtgcccaggccagtacgggatccctgaaccatggaattttccttttcggaggagctactggtgcggacctcggccccccaagagtccagccccttgccccaccccgctggacccaaaggtgctggtagaagaggcaccgcccggcctgagtcctggcgtctccgttcgcagcctggagaagcgctttcctggaagcccgcagccagccctgcgggggctcagcctggacttctaccagggccacatcaccgccttcctgggccacaacggggccggcaagaccaccaccctgtccatcttgagtggcctcttcccacccagtggtggctctgccttcatcctgggccacgacgtccgctccagcatggccgccatccggccccacctgggcgtctgtcctcagtacaacgtgctgtttgacatgctgaccgtggacgagcacgtctggttctatgggcggctgaagggtctgagtgccgctgtagtgggccccgagcaggaccgtctgctgcaggatgtggggctggtctccaagcagagtgtgcagactcgccacctctctggtgggatgcaacggaagctgtccgtggccattgcctttgtgggcggctcccaagttgttatcctggacgagcctacggctggcgtggatcctgcttcccgccgcggtatttgggagctgctgctcaaataccgagaaggtcgcacgctgatcctctccacccaccacctggatgaggcagagctgctgggagaccgtgtggctgtggtggcaggtggccgcttgtgctgctgtggctccccactcttcctgcgccgtcacctgggctccggctactacctgacgctggtgaaggcccgcctgcccctgaccaccaatgagaaggctgacactgacatggagggcagtgtggacaccaggcaggaaaagaagaatggcagccagggcagcagagtcggcactcctcagctgctggccctggtacagcactgggtgcccggggcacggctggtggaggagctgccacacgagctggtgctggtgctgccctacacgggtgcccatgacggcagcttcgccacactcttccgagagctagacacgcggctggcggagctgaggctcactggctacgggatctccgacaccagcctcgaggagatcttcctgaaggtggtggaggagtgtgctgcggacacagatatggaggatggcagctgcgggcagcacctatgcacaggcattgctggcctagacgtaaccctgcggctcaagatgccgccacaggagacagcgctggagaacggggaaccagctgggtcagccccagagactgaccagggctctgggccagacgccgtgggccgggtacagggctgggcactgacccgccagcagctccaggccctgcttctcaagcgctttctgcttgcccgccgcagccgccgcggcctgttcgcccagatcgtgctgcctgccctctttgtgggcctggccctcgtgttcagcctcatcgtgcctcctttcgggcactacccggctctgcggctcagtcccaccatgtacggtgctcaggtgtccttcttcagtgaggacgccccaggggaccctggacgtgcccggctgctcgaggcgctgctgcaggaggcaggactggaggagcccccagtgcagcatagctcccacaggttctcggcaccagaagttcctgctgaagtggccaaggtcttggccagtggcaactggaccccagagtctccatccccagcctgccagtgtagccagcccggtgcccggcgcctgctgcccgactgcccggctgcagctggtggtccccctccgccccaggcagtgaccggctctggggaagtggttcagaacctgacaggccggaacctgtctgacttcctggtcaagacctacccgcgcctggtgcgccagggcctgaagactaagaagtgggtgaatgaggtcaggtacggaggcttctcgctggggggccgagacccaggcctgccctcgggccaagagttgggccgctcagtggaggagttgtgggcgctgctgagtcccctgcctggcggggccctcgaccgtgtcctgaaaaacctcacagcctgggctcacagcctggatgctcaggacagtctcaagatctggttcaacaacaaaggctggcactccatggtggcctttgtcaaccgagccagcaacgcaatcctccgtgctcacctgcccccaggcccggcccgccacgcccacagcatcaccacactcaaccaccccttgaacctcaccaaggagcagctgtctgaggctgcactgatggcctcctcggtggacgtcctcgtctccatctgtgtggtctttgccatgtcctttgtcccggccagcttcactcttgtcctcattgaggagcgagtcacccgagccaagcacctgcagctcatggggggcctgtcccccaccctctactggcttggcaactttctctgggacatgtgtaactacttggtgccagcatgcatcgtggtgctcatctttctggccttccagcagagggcatatgtggcccctgccaacctgcctgctctcctgctgttgctactactgtatggctggtcgatcacaccgctcatgtacccagcctccttcttcttctccgtgcccagcacagcctatgtggtgctcacctgcataaacctctttattggcatcaatggaagcatggccacctttgtgcttgagctcttctctgatcagaagctgcaggaggtgagccggatcttgaaacaggtcttccttatcttcccccacttctgcttgggccgggggctcattgacatggtgcggaaccaggccatggctgatgcctttgagcgcttgggagacaggcagttccagtcacccctgcgctgggaggtggtcggcaagaacctcttggccatggtgatacaggggcccctcttccttctcttcacactactgctgcagcaccgaagccaactcctgccacagcccagggtgaggtctctgccactcctgggagaggaggacgaggatgtagcccgtgaacgggagcgggtggtccaaggagccacccagggggatgtgttggtgctgaggaacttgaccaaggtataccgtgggcagaggatgccagctgttgaccgcttgtgcctggggattccccctggtgagtgttttgggctgctgggtgtgaatggagcagggaagacgtccacgtttcgcatggtgacgggggacacattggccagcaggggcgaggctgtgctggcaggccacagcgtggcccgggaacccagtgctgcgcacctcagcatgggatactgccctcaatccgatgccatctttgagctgctgacgggccgcgagcacctggagctgcttgcgcgcctgcgcggtgtcccggaggcccaggttgcccagaccgctggctcgggcctggcgcgtctgggactctcatggtacgcagaccggcctgcaggcacctacagcggagggaacaaacgcaagctggcgacggccctggcgctggttggggacccagccgtggtgtttctggacgagccgaccacaggcatggaccccagcgcgcggcgcttcctttggaacagccttttggccgtggtgcgggagggccgttcagtgatgctcacctcccatagcatggaggagtgtgaagcgctctgctcgcgcctagccatcatggtgaatgggcggttccgctgcctgggcagcccgcaacatctcaagggcagattcgcggcgggtcacacactgaccctgcgggtgcccgccgcaaggtcccagccggcagcggccttcgtggcggccgagttccctgggtcggagctgcgcgaggcacatggaggccgcctgcgcttccagctgccgccgggagggcgctgcgccctggcgcgcgtctttggagagctggcggtgcacggcgcagagcacggcgtggaggacttttccgtgagccagacgatgctggaggaggtattcttgtacttctccaaggaccaggggaaggacgaggacaccgaagagcagaaggaggcaggagtgggagtggaccccgcgccaggcctgcagcaccccaaacgcgtcagccagttcctcgatgaccctagcactgccgaaactgtgctctgagcctccctcccttgcggggcccgcgggggaggccctggggaatggcaagggcaaggtaaaatgcctangagcccttgaatt

SEQ ID NO: 2 - protein, 2146 aa, Homo sapiens
MAFWTQLMLLLWKNFMYRRRQPVQLLVELLWPLFLFFILVAVRHSHPPLEHHECHFPNKPLPSAGTVPWLQGLICNVNNTCFPQLTPGEEPGRLSNFNDSLVSRLLADARTVLGGASAHRTLAGLGKLIATLRAARSTAQPQPTKQSPLEPPMLDVAELLTSLLRTESLGLALGQAQEPLHSLLEAAEDLAQELLALRSLVELRALLQRPRGTSGPLELLSEALCSVRGPSSTVGPSLNWYEASDLMELVGQEPESALPDSSLSPACSELIGALDSHPLSRLLWRRLKPLILGKLLFAPDTPFTRKLMAQVNRTFEELTLLRDVREVWEMLGPRIFTFMNDSSNVAMLQRLLQMQDEGRRQPRPGGRDHMEALRSFLDPGSGGYSWQDAHADVGHLVGTLGRVTECLSLDKLEAAPSEAALVSRALQLLAEHRFWAGVVFLGPEDSSDPTEHPTPDLGPGHVRIKIRMDIDVVTRTNKIRDRFWDPGPAADPLTDLRYVWGGFVYLQDLVERAAVRVLSGANPRAGLYLQQMPYPCYVDDVFLRVLSRSLPLFLTLAWIYSVTLTVKAVVREKETRLRDTMRAVGLSRAVLWLGWFLSCLGPFLLSAALLVLVLKLGDILPCSHPGVVFLFLAAFAVATVTQSFLLSAFFSRANLAAACGGLAYFSLYLPYVLCVAWRDRLPAGGRVAASLLSPVAFGFGCESLALLEEQGEGAQWHNVGTRPTADVFSLAQVSGLLLLDAALYGLATWYLEAVCPGQYGIPEPWNFPFRRSYWCGPRPPKSPAPCPTPLDPKVLVEEAPPGLSPGVSVRSLEKRFPGSPQPALRGLSLDFYQGHITAFLGHNGAGKTTTLSILSGLFPPSGGSAFILGHDVRSSMAAIRPHLGVCPQYNVLFDMLTVDEHVWFYGRLKGLSAAVVGPEQDRLLQDVGLVSKQSVQTRHLSGGMQRKLSVAIAFVGGSQVVILDEPTAGVDPASRRGIWELLLKYREGRTLILSTHHLDEAELLGDRVAVVAGGRLCCCGSPLFLRRHLGSGYYLTLVKARLPLTTNEKADTDMEGSVDTRQEKKNGSQGSRVGTPQLLALVQHWVPGARLVEELPHELVLVLPYTGAHDGSFATLFRELDTRLAELRLTGYGISDTSLEEIFLKVVEECAADTDMEDGSCGQHLCTGIAGLDVTLRLKMPPQETALENGEPAGSAPETDQGSGPDAVGRVQGWALTRQQLQALLLKRFLLARRSRRGLFAQIVLPALFVGLALVFSLIVPPFGHYPALRLSPTMYGAQVSFFSEDAPGDPGRARLLEALLQEAGLEEPPVQHSSHRFSAPEVPAEVAKVLASGNWTPESPSPACQCSQPGARRLLPDCPAAAGGPPPPQAVTGSGEVVQNLTGRNLSDFLVKTYPRLVRQGLKTKKWVNEVRYGGFSLGGRDPGLPSGQELGRSVEELWALLSPLPGGALDRVLKNLTAWAHSLDAQDSLKIWFNNKGWHSMVAFVNRASNAILRAHLPPGPARHAHSITTLNHPLNLTKEQLSEAALMASSVDVLVSICVVFAMSFVPASFTLVLIEERVTRAKHLQLMGGLSPTLYWLGNFLWDMCNYLVPACIVVLIFLAFQQRAYVAPANLPALLLLLLLYGWSITPLMYPASFFFSVPSTAYVVLTCINLFIGINGSMATFVLELFSDQKLQEVSRILKQVFLIFPHFCLGRGLIDMVRNQAMADAFERLGDRQFQSPLRWEVVGKNLLAMVIQGPLFLLFTLLLQHRSQLLPQPRVRSLPLLGEEDEDVARERERVVQGATQGDVLVLRNLTKVYRGQRMPAVDRLCLGIPPGECFGLLGVNGAGKTSTFRMVTGDTLASRGEAVLAGHSVAREPSAAHLSMGYCPQSDAIFELLTGREHLELLARLRGVPEAQVAQTAGSGLARLGLSWYADRPAGTYSGGNKRKLATALALVGDPAVVFLDEPTTGMDPSARRFLWNSLLAVVREGRSVMLTSHSMEECEALCSRLAIMVNGRFRCLGSPQHLKGRFAAGHTLTLRVPAARSQPAAAFVAAEFPGSELREAHGGRLRFQLPPGGRCALARVFGELAVHGAEHGVEDFSVSQTMLEEVFLYFSKDQGKDEDTEEQKEAGVGVDPAPGLQHPKRVSQFLDDPSTAETVL

SEQ ID NO: 3 - DNA, 5669 bp, Homo sapiens
atggccttctggacacagctgatgctgctgctctggaagaatttcatgtatcgccggagacagccggtccagctcctggtcgaattgctgtggcctctcttcctcttcttcatcctggtggctgttcgccactcccacccgcccctggagcaccatgaatgccacttcccaaacaagccactgccatcggcgggcaccgtgccctggctccagggtctcatctgtaatgtgaacaacacctgctttccgcagctgacaccgggcgaggagcccgggcgcctgagcaacttcaacgactccctggtctcccggctgctagccgatgcccgcactgtgctgggaggggccagtgcccacaggacgctggctggcctagggaagctgatcgccacgctgagggctgcacgcagcacggcccagcctcaaccaaccaagcagtctccactggaaccacccatgctggatgtcgcggagctgctgacgtcactgctgcgcacggaatccctggggttggcactgggccaagcccaggagcccttgcacagcttgttggaggccgctgaggacctggcccaggagctcctggcgctgcgcagcctggtggagcttcgggcactgctgcagagaccccgagggaccagcggccccctggagttgctgtcagaggccctctgcagtgtcaggggacctagcagcacagtgggcccctccctcaactggtacgaggctagtgacctgatggagctggtggggcaggagccagaatccgccctgccagacagcagcctgagccccgcctgctcggagctgattggagccctggacagccacccgctgtcccgcctgctctggagacgcctgaagcctctgatcctcgggaagctactctttgcaccagatacaccttttacccggaagctcatggcccaggtgaaccggaccttcgaggagctcaccctgctgagggatgtccgggaggtgtgggagatgctgggaccccggatcttcaccttcatgaacgacagttccaatgtggccatgctgcagcggctcctgcagatgcaggatgaaggaagaaggcagcccagacctggaggccgggaccacatggaggccctgcgatcctttctggaccctgggagcggtggctacagctggcaggacgcacacgctgatgtggggcacctggtgggcacgctgggccgagtgacggagtgcctgtccttggacaagctggaggcggcaccctcagaggcagccctggtgtcgcgggccctgcaactgctcgcggaacatcgattctgggccggcgtcgtcttcttgggacctgaggactcttcagaccccacagagcacccaaccccagacctgggccccggccacgtgcgcatcaaaatccgcatggacattgacgtggtcacgaggaccaataagatcagggacaggttttgggaccctggcccagccgcggaccccctgaccgacctgcgctacgtgtggggcggcttcgtgtacctgcaagacctggtggagcgtgcagccgtccgcgtgctcagcggcgccaacccccgggccggcctctacctgcagcagatgccctatccgtgctatgtggacgacgtgttcctgcgtgtgctgagccggtcgctgccgctcttcctgacgctggcctggatctactccgtgacactgacagtgaaggccgtggtgcgggagaaggagacgcggctgcgggacaccatgcgcgccgtggggctcagccgcgcggtgctctggctaggctggttcctcagctgcctcgggcccttcctgctcagcgccgcgctgctggttctggtgctcaagctgggggacatcctcccctgcagccacccgggcgtggtcttcctgttcttggcagccttcgcggtggccacggtgacccagagcttcctgctcagcgccttcttctcccgcgccaacctggctgcggcctgcggcggcctggcctacttctccctctacctgccctacgtgctgtgtgtggcttggcgggaccggctgcccgcgggtggccgcgtggccgcgagcctgctgtcgcccgtggccttcggcttcggctgcgagagcctggctctgctggaggagcagggcgagggcgcgcagtggcacaacgtgggcacccggcctacggcagacgtcttcagcctggcccaggtctctggccttctgctgctggacgcggcgctctacggcctcgccacctggtacctggaagctgtgtgcccaggccagtacgggatccctgaaccatggaattttccttttcggaggagctactggtgcggacctcggccccccaagagtccagccccttgccccaccccgctggacccaaaggtgctggtagaagaggcaccgcccggcctgagtcctggcgtctccgttcgcagcctggagaagcgctttcctggaagcccgcagccagccctgcgggggctcagcctggacttctaccagggccacatcaccgccttcctgggccacaacggggccggcaagaccaccaccctgtccatcttgagtggcctcttcccacccagtggtggctctgccttcatcctgggccacgacgtccgctccagcatggccgccatccggccccacctgggcgtctgtcctcagtacaacgtgctgtttgacatgctgaccgtggacgagcacgtctggttctatgggcggctgaagggtctgagtgccgctgtagtgggccccgagcaggaccgtctgctgcaggatgtggggctggtctccaagcagagtgtgcagactcgccacctctctggtgggatgcaacggaagctgtccgtggccattgcctttgtgggcggctcccaagttgttatcctggacgagcctacggctggcgtggatcctgcttcccgccgcggtatttgggagctgctgctcaaataccgagaaggtcgcacgctgatcctctccacccaccacctggatgaggcagagctgctgggagaccgtgtggctgtggtggcaggtggccgcttgtgctgctgtggctccccactcttcctgcgccgtcacctgggctccggctactacctgacgctggtgaaggcccgcctgcccctgaccaccaatgagaaggctgacactgacatggagggcagtgtggacaccaggcaggaaaagaagaatggcagccagggcagcagagtcggcactcctcagctgctggccctggtacagcactgggtgcccggggcacggctggtggaggagctgccacacgagctggtgctggtgctgccctacacgggtgcccatgacggcagcttcgccacactcttccgagagctagacacgcggctggcggagctgaggctcactggctacgggatctccgacaccagcctcgaggagatcttcctgaaggtggtggaggagtgtgctgcggacacagatatggaggatggcagctgcgggcagcacctatgcacaggcattgctggcctagacgtaaccctgcggctcaagatgccgccacaggagacagcgctggagaacggggaaccagctgggtcagccccagagactgaccagggctctgggccagacgccgtgggccgggtacagggctgggcactgacccgccagcagctccaggccctgcttctcaagcgctttctgcttgcccgccgcagccgccgcggcctgttcgcccagatcgtgctgcctgccctctttgtgggcctggccctcgtgttcagcctcatcgtgcctcctttcgggcactacccggctctgcggctcagtcccaccatgtacggtgctcaggtgtccttcttcagtgaggacgccccaggggaccctggacgtgcccggctgctcgaggcgctgctgcaggaggcaggactggaggagcccccagtgcagcatagctcccacaggttctcggcaccagaagttcctgctgaagtggccaaggtcttggccagtggcaactggaccccagagtctccatccccagcctgccagtgtagccagcccggtgcccggcgcctgctgcccgactgcccggctgcagctggtggtccccctccgccccaggcagtgaccggctctggggaagtggttcagaacctgacaggccggaacctgtctgacttcctggtcaagacctacccgcgcctggtgcgccagggcctgaagactaagaagtgggtgaatgaggtcaggtacggaggcttctcgctggggggccgagacccaggcctgccctcgggccaagagttgggccgctcagtggaggagttgtgggcgctgctgagtcccctgcctggcggggccctcgaccgtgtcctgaaaaacctcacagcctgggctcacagcctggatgctcaggacagtctcaagatctggttcaacaacaaaggctggcactccatggtggcctttgtcaaccgagccagcaacgcaatcctccgtgctcacctgcccccaggcccggcccgccacgcccacagcatcaccacactcaaccaccccttgaacctcaccaaggagcagctgtctgaggctgcactgatggcctcctcggtggacgtcctcgtctccatctgtgtggtctttgccatgtcctttgtcccggccagcttcactcttgtcctcattgaggagcgagtcacccgagccaagcacctgcagctcatggggggcctgtcccccaccctctactggcttggcaactttctctgggacatgtgtaactacttggtgccagcatgcatcgtggtgctcatctttctggccttccagcagagggcatatgtggcccctgccaacctgcctgctctcctgctgttgctactactgtatggctggtcgatcacaccgctcatgtacccagcctccttcttcttctccgtgcccagcacagcctatgtggtgctcacctgcataaacctctttattggcatcaatggaagcatggccacctttgtgcttgagctcttctctgatcagaagctgcaggaggtgagccggatcttgaaacaggtcttccttatcttcccccacttctgcttgggccgggggctcattgacatggtgcggaaccaggccatggctgatgcctttgagcgcttgggagacaggcagttccagtcacccctgcgctgggaggtggtcggcaagaacctcttggccatggtgatacaggggcccctcttccttctcttcacactactgctgcagcaccgaagccaactcctgccacagcccagggtgaggtctctgccactcctgggagaggaggacgaggatgtagcccgtgaacgggagcgggtggtccaaggagccacccagggggatgtgttggtgctgaggaacttgaccaaggtataccgtgggcagaggatgccagctgttgaccgcttgtgcctggggattccccctggtgaggtgagtccaggggtggaggccaggtgcagggacagtgagtggctgccctactgcatgccctgcccatcatcctttactgaacacctactgtgtatccaccaccttttattgggcacctactgtatgccaatatttgtgctcctatttttattttattaaattattatttatttaaaaaaaaaaaaaaaaaaaaaaaaaaaaaaaaa

SEQ ID NO: 4 - protein, 1873 aa, Homo sapiens
MAFWTQLMLLLWKNFMYRRRQPVQLLVELLWPLFLFFILVAVRHSHPPLEHHECHFPNKPLPSAGTVPWLQGLICNVNNTCFPQLTPGEEPGRLSNFNDSLVSRLLADARTVLGGASAHRTLAGLGKLIATLRAARSTAQPQPTKQSPLEPPMLDVAELLTSLLRTESLGLALGQAQEPLHSLLEAAEDLAQELLALRSLVELRALLQRPRGTSGPLELLSEALCSVRGPSSTVGPSLNWYEASDLMELVGQEPESALPDSSLSPACSELIGALDSHPLSRLLWRRLKPLILGKLLFAPDTPFTRKLMAQVNRTFEELTLLRDVREVWEMLGPRIFTFMNDSSNVAMLQRLLQMQDEGRRQPRPGGRDHMEALRSFLDPGSGGYSWQDAHADVGHLVGTLGRVTECLSLDKLEAAPSEAALVSRALQLLAEHRFWAGVVFLGPEDSSDPTEHPTPDLGPGHVRIKIRMDIDVVTRTNKIRDRFWDPGPAADPLTDLRYVWGGFVYLQDLVERAAVRVLSGANPRAGLYLQQMPYPCYVDDVFLRVLSRSLPLFLTLAWIYSVTLTVKAVVREKETRLRDTMRAVGLSRAVLWLGWFLSCLGPFLLSAALLVLVLKLGDILPCSHPGVVFLFLAAFAVATVTQSFLLSAFFSRANLAAACGGLAYFSLYLPYVLCVAWRDRLPAGGRVAASLLSPVAFGFGCESLALLEEQGEGAQWHNVGTRPTADVFSLAQVSGLLLLDAALYGLATWYLEAVCPGQYGIPEPWNFPFRRSYWCGPRPPKSPAPCPTPLDPKVLVEEAPPGLSPGVSVRSLEKRFPGSPQPALRGLSLDFYQGHITAFLGHNGAGKTTTLSILSGLFPPSGGSAFILGHDVRSSMAAIRPHLGVCPQYNVLFDMLTVDEHVWFYGRLKGLSAAVVGPEQDRLLQDVGLVSKQSVQTRHLSGGMQRKLSVAIAFVGGSQVVILDEPTAGVDPASRRGIWELLLKYREGRTLILSTHHLDEAELLGDRVAVVAGGRLCCCGSPLFLRRHLGSGYYLTLVKARLPLTTNEKADTDMEGSVDTRQEKKNGSQGSRVGTPQLLALVQHWVPGARLVEELPHELVLVLPYTGAHDGSFATLFRELDTRLAELRLTGYGISDTSLEEIFLKVVEECAADTDMEDGSCGQHLCTGIAGLDVTLRLKMPPQETALENGEPAGSAPETDQGSGPDAVGRVQGWALTRQQLQALLLKRFLLARRSRRGLFAQIVLPALFVGLALVFSLIVPPFGHYPALRLSPTMYGAQVSFFSEDAPGDPGRARLLEALLQEAGLEEPPVQHSSHRFSAPEVPAEVAKVLASGNWTPESPSPACQCSQPGARRLLPDCPAAAGGPPPPQAVTGSGEVVQNLTGRNLSDFLVKTYPRLVRQGLKTKKWVNEVRYGGFSLGGRDPGLPSGQELGRSVEELWALLSPLPGGALDRVLKNLTAWAHSLDAQDSLKIWFNNKGWHSMVAFVNRASNAILRAHLPPGPARHAHSITTLNHPLNLTKEQLSEAALMASSVDVLVSICVVFAMSFVPASFTLVLIEERVTRAKHLQLMGGLSPTLYWLGNFLWDMCNYLVPACIVVLIFLAFQQRAYVAPANLPALLLLLLLYGWSITPLMYPASFFFSVPSTAYVVLTCINLFIGINGSMATFVLELFSDQKLQEVSRILKQVFLIFPHFCLGRGLIDMVRNQAMADAFERLGDRQFQSPLRWEVVGKNLLAMVIQGPLFLLFTLLLQHRSQLLPQPRVRSLPLLGEEDEDVARERERVVQGATQGDVLVLRNLTKVYRGQRMPAVDRLCLGIPPGEVSPGVEARCRDSEWLPYCMPCPSSFTEHLLCIHHLLLGTYCMPIFVLLFLFY

SEQ ID NO: 5 - protein, 1800 aa, Homo sapiens
  UNSURE (115): Any
MPSAGTLPWVQGIICNANNPCFRYPTPGEAPGVVGNFNKSIVSRLFSDAQRLLLYSQRDTSIKDMHKVLRMLRQIKHPNSNLKLQDFLVDNETFSGFLQHNLSLPRSTVDSLLQXNVGLQKVFLQGYQLHLASLCNGSKLEEIIQLGDAEVSALCGLPRKKLDAAERVLRYNMDILKPVVTKLNSTSHLPTQHLAEATTVLLDSLGGLAQELFSTKSWSDMRQEVMFLTNVNSSSSSTQIYQAVSRIVCGHPEGGGLKIKSLNWYEDNNYKALFGGNNTEEDVDTFYDNSTTPYCNDLMKNLESSPLSRIIWKALKPLLVGKILYTPDTPATRQVMAEVNKTFQELAVFHDLEGMWEELSPQIWTFMENSQEMDLVRTLLDSRGNDQFWEQKLDGLDWTAQDIMAFLAKNPEDVQSPNGSVYTWREAFNETNQAIQTISRFMECVNLNKLEPIPTEVRLINKSMELLDERKFWAGIVFTGITPDSVELPHHVKYKIRMDIDNVERTNKIKDGYWDPGPRADPFEDMRYVWGGFAYLQDVVEQAIIRVLTGSEKKTGVYVQQMPYPCYVDDIFLRVMSRSMPLFMTLAWIYSVAVIIKSIVYEKEARLKETMRIMGLDNGILWFSWFVSSLIPLLVSAGLLVVILKLGNLLPYSDPSVVFVFLSVFAMVTILQCFLISTLFSRANLAAACGGIIYFTLYLPYVLCVAWQDYVGFSIKIFASLLSPVAFGFGCEYFALFEEQGIGVQWDNLFESPVEEDGFNLTTAVSMMLFDTFLYGVMTWYIEAVFPGQYGIPRPWYFPCTKSYWFGEEIDEKSHPGSSQKGVSEICMEEEPTHLRLGVSIQNLVKVYRDGMKVAVDGLALNFYEGQITSFLGHNGAGKTTTMSILTGLFPPTSGTAYILGKDIRSEMSSIRQNLGVCPQHNVLFDMLTVEEHIWFYARLKGLSEKHVKAEMEQMALDVGLPPSKLKSKTSQLSGGMQRKLSVALAFVGGSKVVILDEPTAGVDPYSRRGIWELLLKYRQGRTIILSTHHMDEADILGDRIAIISHGKLCCVGSSLFLKNQLGTGYYLTLVKKDVESSLSSCRNSSSTVSCLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLHPFTEDDAVDPNDSDIDPESRETDLLSGMDGKGSYQLKGWKLTQQQFVALLWKRLLIARRSRKGFFAQIVLPAVFVCIALVFSLIVPPFGKYPSLELQPWMYNEQYTFVSNDAPEDMGTQELLNALTKDPGFGTRCMEGNPIPDTPCLAGEEDWTISPVPQSIVDLFQNGNWTMKNPSPACQCSSDKIKKMLPVCPPGAGGLPPPQRKQKTADILQNLTGRNISDYLVKTYVQIIAKSLKNKIWVNEFRYGGFSLGVSNSQALPPSHEVNDAIKQMKKLLKLTKDTSADRFLSSLGRFMAGLDTKNNVKVWFNNKGWHAISSFLNVINNAILRANLQKGENPSQYGITAFNHPLNLTKQQLSEVALMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTNNKLNDTNDILKSVFLIFPHFCLGRGLIDMVKNQAMADALERFGENRFVSPLSWDLVGRNLFAMAVEG

SEQ ID NO: 6 - protein, 2273 aa, Homo sapiens [sequence omitted]

SEQ ID NO: 7 - DNA, 25 bp, Artificial Sequence
  Description of Artificial Sequence primer
tctcaccatggccttctggacacag

SEQ ID NO: 8 - DNA, 23 bp, Artificial Sequence
  Description of Artificial Sequence primer
cacgtagcgcaggtcggtcaggg

SEQ ID NO: 9 - DNA, 24 bp, Artificial Sequence
  Description of Artificial Sequence primer
gctgattggagccctggacagcca

SEQ ID NO: 10 - DNA, 24 bp, Artificial Sequence
  Description of Artificial Sequence primer
gtccacatagcacggatagggcat

SEQ ID NO: 11 - DNA, 23 bp, Artificial Sequence
  Description of Artificial Sequence primer
tcgtgtacctgcaagacctggtg

SEQ ID NO: 12 - DNA, 21 bp, Artificial Sequence
  Description of Artificial Sequence primer
cagagccaggctctcgcagcc

SEQ ID NO: 13 - DNA, 25 bp, Artificial Sequence
  Description of Artificial Sequence primer
cagcttcactcttgtcctcattgag

SEQ ID NO: 14 - DNA, 24 bp, Artificial Sequence
  Description of Artificial Sequence primer
tttatgcaggtgagcaccacatag

SEQ ID NO: 15 - DNA, 25 bp, Artificial Sequence
  Description of Artificial Sequence primer
cccctcttccttctcttcacactac

SEQ ID NO: 16 - DNA, 22 bp, Artificial Sequence
  Description of Artificial Sequence primer
agcagcccaaaacactcaccac

SEQ ID NO: 17 - DNA, 24 bp, Artificial Sequence
  Description of Artificial Sequence primer
tgggagaggaggacgaggatgtag

SEQ ID NO: 18 - DNA, 24 bp, Artificial Sequence
  Description of Artificial Sequence primer
aggtgttcagtaaaggatgatggg